(19) United States
(12) Patent Application Publication
(10) Pub. No.: US 2007/0243167 A1
(43) Pub. Date: Oct. 18, 2007

(54) Novel Herpes Simplex Viruses

(76) Inventors: Susanne Moira Brown, Glasgow (GB); Joe Conner, Glasgow (GB)

Correspondence Address: KLARQUIST SPARKMAN, LLP, 121 SW SALMON STREET, SUITE 1600, PORTLAND, OR, 97204, US

(21) Appl. No.: 11/547,951

(22) PCT Filed: Apr. 5, 2005
(86) PCT No.: PCT/GB05/01313

§ 371 Date: Oct. 5, 2006

(30) Foreign Application Priority Data
Apr. 8, 2004 (GB) 0408103.0

(51) Int. Cl. A61K 48/00; C12N 7/00; C12N 15/86; A61K 38/28; C07K 14/62

(52) U.S. Cl. 424/93.2; 435/235.1; 435/456; 530/303

(57) ABSTRACT

An herpes simplex virus wherein the herpes simplex virus genome comprises nucleic acid encoding insulin is disclosed. Viruses disclosed are capable of expressing insulin. They may lack neurovirulence and may find use in the treatment of diseases involving abnormal insulin expression or function, such as diabetes.

### FIELD OF THE INVENTION

The present invention relates to herpes simplex viruses comprising an exogenous nucleic acid sequence.

### BACKGROUND TO THE INVENTION

Insulin

Insulin is an anabolic signalling molecule having a role in several biochemical pathways. Binding of insulin to its receptor is known to initiate a series of events resulting in the increased uptake of glucose into cells. In addition to having a role in controlling cellular glucose levels insulin is also implicated in the transport of amino acids across cell membranes, lipolysis and glycogen synthase activity.

Abnormal insulin expression or function is implicated in disease states, notably in forms of insulin dependent diabetes mellitus (Type I diabetes).

Insulin is normally produced by B-cells in the islets of Langerhans of the pancreas. Translation results in a precursor molecule known as preproinsulin comprising a single polypeptide of three segments—A, B and C. Preproinsulin subsequently undergoes structural processing with the formation of disulphide bonds between the A and B segments to form proinsulin. The secreted hormone insulin is the result of a further processing step in which the C peptide, linking the A and B peptides, is cleaved.

The sequence and structure of the insulin gene and protein has been well characterised. Sequence information is available from the NCBI database (http://www.ncbi.nlm.nih.gov/). For example, the human insulin gene sequence can be accessed under accession number AH002844 (GI:186429) and the mRNA sequence and amino acid sequence for human insulin can be accessed from the same database under accession number NM_{—}000207 (GI:4557670).

Insulin dependent diabetes can be treated by regular insulin injections. However, this requires careful monitoring of blood glucose levels and repeated injections and thus carries with it considerable inconvenience and possibly danger to the patient if a supply of insulin is not available.

Introduction of the insulin gene to the patient by ‘gene therapy’ techniques also presents problems. For example a vector is required which stably delivers the gene and enables the necessary transcription, translation and post-translational processing to take place.

Herpes Simplex Virus (HSV)

The genomes of the two herpes simplex virus serotypes, HSV-1 and HSV-2, have been well characterised and genomic sequence information is available for a number of strains (e.g. the sequence of the HSV-1 strain 17 long repeat regions³ or the HSV-2 strain HG52 complete genome sequence which is available under accession number NC_{—}001798 (GI:9629267) from the NCBI database). Given the high degree of characterisation, both HSV-1 and HSV-2 genomes can be manipulated by known genetic engineering techniques.

The HSV genome comprises two covalently linked segments, designated long (L) and short (S). Each segment contains a unique sequence flanked by a pair of inverted repeat sequences. The long repeat (R_{L}) and the short repeat (R_{S}) are distinct. The long repeats are sometimes referred to by their respective positions in the HSV genome, i.e. as the internal long repeat (IR_{L}) and terminal long repeat (TR_{L}).

The HSV ICP34.5 (also γ34.5) gene, which has been extensively studied^{1,6,7,8}, has been sequenced in HSV-1 strains F⁹ and syn17+³ and in HSV-2 strain HG52⁴. One copy of the ICP34.5 gene is in the RL1 locus of each long repeat (R_{L}). Mutants inactivating both copies of the ICP34.5 gene (i.e. null mutants), e.g. HSV-1 strain 17162 or the mutants R3616 or R4009 in strain F⁵, are known to lack neurovirulence, i.e. be avirulent, and have utility in the treatment of tumours by oncolysis. HSV strain 1716 has a 759 bp deletion in each copy of the ICP34.5 gene located within the BamHI s restriction fragment of each RL repeat.

HSV is capable of infecting a wide variety of cell types, including dividing and non-dividing tissues but does not normally replicate in vivo in non-neuronal peripheral tissues. Cellular infection with HSV does not result in integration of the HSV DNA into the genome of the host cell. Infection with virulent HSV usually leads to peripheral and central nervous system infections, which in the case of many herpes simplex virus strains results in a virulent encephalitis with serious damage to the nervous system followed by death of the patient. Hence, herpes simplex has not been considered a suitable vector for gene therapy applications.

### SUMMARY OF THE INVENTION

The inventors have now determined that HSV having an inactivating mutation in the RL1 locus, more specifically a mutation which inactivates the function of the ICP34.5 gene product, such that the HSV is non-neurovirulent can be used as gene therapy vectors⁶. Non-neurovirulence is defined by the ability to introduce a high titre of virus (approx 10⁶ plaque forming units (pfu)) to an animal or patient^{10,11,12} without causing a lethal encephalitis such that the LD₅₀ in animals, e.g. mice, or human patients is in the approximate range of ≧10⁶ pfu (ref 13)).

Moreover, the inventors have shown that genetically engineered non-neurovirulent HSV, the DNA of which encodes the sequence of human preproinsulin under the control of a constitutive or inducible promoter, can be used to express preproinsulin and proinsulin and obtain cleavage of the C-peptide, i.e. the mature insulin form is obtained, in a range of cell types.

The inventors have also provided a genetically engineered non-neurovirulent HSV, wherein the genomic DNA of the virus encodes a mutant form of human insulin. In this mutant a His residue, normally located at position 10 of the B-chain, is mutated to an Asp residue. This mutation has been reported to increase biopotency. The mutant also incorporates the tetrapeptide motifs RTRR and RQKR. These provide sites for cleavage of the immature polypeptide by the ubiquitous furin protease to form the active mature form of the insulin protein. Accordingly, this enables active forms of insulin to be obtained by infection with the HSV of cell types in which insulin specific peptidases are not present such that the preproinsulin and/or proinsulin, which would otherwise remain unprocessed, can be processed to the mature and active forms.

The inventors have taken this further by placing the DNA encoding the mutant form of human insulin under the control of the human metallothionein promoter and a carbohydrate response element. This enables the transcription, and thereby the expression, of the HSV encoded insulin to be regulated, at least in part, by the available metal ion and/or carbohydrate levels. In particular, this provides a feedback mechanism by which changes (e.g. an increase or decrease) in carbohydrate or metal ion concentrations may activate or repress expression of the HSV encoded insulin. These response elements may be responsive to metal ions such as zinc (Zn²⁺) or carbohydrates such as glucose or fructose.

At its most general the present invention comprises a herpes simplex virus, wherein the herpes simplex genome comprises an exogenous nucleic acid sequence.

Herpes simplex viruses according to the present invention are preferably non-neurovirulent.

According to a first aspect of the present invention there is provided a herpes simplex virus, wherein the genome of said virus comprises an exogenous nucleic acid sequence in at least one of the long repeat regions (R_{L}).

According to a second aspect of the present invention there is provided a herpes simplex virus, wherein the genome of said virus comprises an exogenous nucleic acid sequence in at least one of the long repeat regions (R_{L}) for use in the treatment of a disease.

According to a third aspect of the present invention there is provided the use of a herpes simplex virus, wherein the genome of said virus comprises an exogenous nucleic acid sequence in at least one of the long repeat regions (R_{L}), in the manufacture of a medicament for the treatment of a disease.

In a fourth aspect of the present invention there is provided a method for the treatment of a disease comprising the step of administering to a patient in need of treatment a herpes simplex virus, wherein the genome of said virus comprises an exogenous nucleic acid sequence in at least one of the long repeat regions (R_{L}).

According to a fifth aspect of the present invention there is provided a method of expressing in vitro or in vivo one or more of preproinsulin, proinsulin or insulin, said method comprising the step of infecting at least one cell or tissue of interest with a non-neurovirulent herpes simplex virus, wherein the genome of said virus comprises a nucleic acid sequence encoding a preproinsulin.

According to a sixth aspect of the present invention there is provided an in vitro or in vivo method of delivering an exogenous gene or protein coding sequence to at least one cell or to a tissue of interest said method comprising the step of infecting said cell(s) or tissue with a non-neurovirulent herpes simplex virus, wherein the genome of said virus comprises a nucleic acid sequence encoding said gene or protein coding sequence.

Preferably, the herpes simplex virus contains at least one copy of the exogenous nucleic acid sequence in each long repeat region (R_{L}), i.e. in the terminal and internal long repeat (TR_{L} and IR_{L}) regions. In a particularly preferred arrangement each exogenous sequence is located in the DNA encoding the ICP34.5 gene of the herpes simplex virus. The herpes simplex virus is thereby non-neurovirulent.

The parent herpes simplex virus, from which a virus of the invention is derived may be of any kind, e.g. HSV-1 or HSV-2. In one preferred arrangement the herpes simplex virus is a variant of HSV-1 strain 17 and may be obtained by modification of the strain 17 genomic DNA. Suitable modifications include the insertion of the exogenous nucleic acid sequence into the herpes simplex virus genomic DNA. The insertion may be performed by homologous recombination of the exogenous nucleic acid sequence into the genome of the selected herpes simplex virus.

Although the non-neurovirulent phenotype of the herpes simplex virus of the invention may be the result of insertion of the exogenous nucleic acid sequence in the RL1 locus, in an alternative aspect of the invention herpes simplex viruses according to the present invention may be obtained by utilising a non-neurovirulent parent strain, e.g. HSV1716 deposited at the European Collection of Animal Cell Cultures (ECACC), Porton Down, Salisbury, Wiltshire, United Kingdom under accession number V92012803, and inserting the exogenous nucleic acid sequence at another location of the genome by standard genetic engineering techniques, e.g. homologous recombination. In this aspect the location selected for insertion of the exogenous nucleic acid sequence may be a neutral location.

Herpes simplex viruses of the present invention may be mutants or variants of a known ‘parent’ strain from which the herpes simplex virus of the invention has been derived. A particularly preferred parent strain is HSV-1 strain 17. Other parent strains may include HSV-1 strain F or HSV-2 strain HG52. A variant comprises an HSV in which the genome substantially resembles that of the parent, contains the exogenous nucleic acid sequence and may contain a limited number of other modifications, e.g. one, two or three other specific mutations, which may be introduced to disable the pathogenic properties of the herpes simplex virus, for example a mutation in the ribonucleotide reductase (RR) gene, the 65K trans inducing factor (aTIF) and/or a small number of mutations resulting from natural variation, which may be incorporated naturally during replication and selection in vitro or in vivo. Otherwise the genome of the variant will be that of the parent strain.

The exogenous nucleic acid sequence is a sequence not originating in the herpes simplex virus and preferably comprises nucleic acid (more preferably DNA) encoding a form of insulin, more preferably a form of preproinsulin or proinsulin. The sequence of the form of insulin may be derived or obtained from any animal including humans and non-human mammals and may be selected from those sequences which are publicly available.

Thus, according to one aspect of the present invention there is provided an herpes simplex virus wherein the herpes simplex virus genome comprises nucleic acid encoding insulin.

The insulin may be human insulin and may be encoded by the nucleic acid of SEQ ID No.2 or SEQ ID No.5 or by nucleic acid encoding the polypeptide of SEQ ID No.1 or SEQ ID No.6. Alternatively, the nucleic acid may have at least 60%, 70%, 80%, 85%, 86%, 87%, 88%, 89%, 90%, 91%, 92%, 93%, 94%, 95%, 96%, 97%, 98% or 99% sequence identity to SEQ ID No.2 or SEQ ID No.5 or to a nucleic acid encoding the polypeptide of SEQ ID No.1 or 6. In a further alternative arrangement the nucleic acid may hybridise to the nucleic acid of SEQ ID No.2 or SEQ ID No.5, to the complement of SEQ ID No.2 or SEQ ID No.5 or to a nucleic acid encoding the polypeptide of SEQ ID No.1 or SEQ ID No.6 under high stringency conditions. The nucleic acid may encode a modified insulin polypeptide which incorporates one or more predetermined peptide motifs which are cleavage sites for a selected protease. The selected protease may be one that does not cleave at least one of wild type preproinsulin, proinsulin or insulin.

The predetermined peptide motif may be a cleavage site for a protease present in cells which do not normally produce insulin and which may be infected by the herpes simplex virus and may be formed by modification of a protease cleavage site of wild type insulin. For example, the peptide motif may be formed by modification of the amino acid sequence of wild type insulin at which the C-peptide is cleaved. Another suitable peptide motif may be formed by modification of the amino acid sequence of wild type insulin at which the proinsulin polypeptide is cleaved into chains A and B.

The selected protease may be furin protease. And may recognise one or both of the peptide motifs RTRR and RQKR.

The insulin encoding nucleic acid is preferably located in at least one RL1 locus of the herpes simplex virus genome. More preferably, the nucleic acid is located in, or overlaps, at least one of the ICP34.5 protein coding sequences of the herpes simplex virus genome.

The herpes simplex virus may be a gene specific null mutant, more preferably an ICP34.5 null mutant. In the latter both copies of the ICP34.5 gene may be mutated such that the virus cannot express a normally functional ICP34.5 protein.

The herpes simplex virus may lack at least one expressible ICP34.5 gene or may lack only one expressible ICP34.5 gene.

The herpes simplex virus is preferably non-neurovirulent.

In a further aspect of the present invention there is provided an herpes simplex virus, wherein the genome of said virus comprises a nucleic acid sequence encoding insulin in at least one of the long repeat regions, (R_{L}).

In yet a further aspect of the present invention there is provided an herpes simplex virus, wherein the genome of said virus comprises a nucleic acid sequence encoding insulin and wherein the herpes simplex virus is non-neurovirulent.

Herpes simplex virus according to the present invention may be provided for use in a method of medical treatment, such as treatment of a disease state. Use of an herpes simplex virus in the manufacture of a medicament for treatment of disease is also provided.

In another aspect of the present invention there is provided a method of expressing one or more of preproinsulin, proinsulin and/or insulin in vitro or in vivo comprising the step of administering to a patient in need of treatment an herpes simplex virus according to the present invention.

In yet a further aspect of the present invention there is provided a method of expressing insulin in vitro or in vivo, said method comprising the step of infecting at least one cell or tissue of interest with a herpes simplex virus, wherein the genome of said virus comprises a nucleic acid sequence encoding insulin in at least one of the long repeat regions (R_{L}), said insulin operably linked to a transcription regulatory sequence.

In yet another aspect of the present invention there is provided a method of expressing insulin in vitro or in vivo, said method comprising the step of infecting at least one cell or tissue of interest with a non-neurovirulent herpes simplex virus, wherein the genome of said virus comprises a nucleic acid sequence encoding insulin, said insulin operably linked to a transcription regulatory sequence.

A cell or cells provided in vitro, preferably as part of a cell culture, infected with an herpes simplex virus according to the present invention is/are also provided.

The exogenous nucleic acid sequence preferably further comprises a constitutive or inducible control sequence, e.g. enhancer and/or promoter sequence (e.g. the cytomegalovirus promoter (constitutive) or glucose sensitive promoters such as a native glucose responsive insulin promoter or the pyruvate kinase promoter or human metallothionein IIA promoter (both inducible)), 5′ (upstream) of the preproinsulin transcription initiation site. One suitable promoter is the constitutive cytomegalovirus (CMV) promoter. A polyadenylation (polyA) sequence, e.g. the Simian Virus 40 (SV40) polyA sequence or the polyA sequence contained within the native 3′ untranslated region of the preproinsulin gene may be located 3′ (downstream) of the preproinsulin coding sequence. The exogenous nucleic acid sequence may thus form a nucleic acid cassette which is inserted in the RL1 locus or other genomic location selected. Most preferably it is inserted in each ICP34.5 protein coding sequence of the HSV genomic DNA to produce a modified virus which is a non-neurovirulent mutant capable of expressing preproinsulin upon transfection into mammalian, more preferably human, cells in vivo and in vitro in a form which can be processed by the cellular machinery to provide the active ‘mature’ form of insulin, which is preferably secreted from the cell.

Inducible regulatory nucleotide sequences may be used to regulate insulin expression in response to a change in the concentration of one or more selected metal ions, e.g. zinc ions. Additionally, or alternatively, a regulatory sequence which is inducible in response to a change in the concentration of one or more selected carbohydrates (e.g. glucose or fructose) may be incorporated. This may comprise a carbohydrate response element, for example the element of SEQ ID No.7 or an element having substantial sequence identity thereto, e.g. one having at least 80%, 85%, 86%, 87%, 88%, 89%, 90%, 91%, 92%, 93%, 94%, 95%, 96%, 97%, 98% or 99% sequence identity. One or a plurality of such carbohydrate response elements may be incorporated.

The HSV may also encode a marker nucleic acid, e.g. nucleic acid encoding the green fluorescent protein. This may be used to determine successful transformation of the virus with the selected exogenous nucleic acid. The marker may be a defined nucleotide sequence encoding a polypeptide, e.g. the Green Fluorescent Protein (GFP) or the enhanced Green Fluorescent Protein (EGFP). Alternatively the marker may comprise a defined nucleotide sequence detectable by hybridisation under high stringency conditions with a corresponding labelled nucleic acid probe. The marker nucleic acid may be operably linked to a regulatory sequence.

In one arrangement the insulin nucleic acid and marker nucleic acid are separated by an internal ribosome entry site (IRES) such as the encephalomyocarditis virus IRES. This permits the production of a bi- or poly-cistronic transcript comprising a first cistron encoding insulin and a second cistron encoding the marker, wherein the ribosome binding site is preferably located between said first and second cistrons. By operably linking a nucleic acid cassette containing the insulin nucleic acid and marker nucleic acid separated by the IRES to a regulatory sequence a bicistronic transcript containing RNA encoding preproinsulin and the marker nucleic acid can be obtained. The resultant expression and detection of the marker nucleic acid (or the protein it encodes) directly indicates expression of insulin by the respective HSV.

Accordingly, the nucleic acid encoding insulin may form part of a nucleic acid cassette integrated in the genome of the herpes simplex virus, said cassette encoding:
(a) nucleic acid encoding insulin; and nucleic acid encoding
(b) a ribosome binding site; and
(c) a marker,
   wherein the nucleic acid encoding insulin is arranged upstream (5′) of the ribosome binding site and the ribosome binding site is arranged upstream (5′) of the marker. A regulatory nucleotide sequence may be located upstream (5′) of the nucleic acid encoding insulin, wherein the regulatory nucleotide sequence has a role in regulating transcription of said nucleic acid encoding insulin. Preferably, the cassette disrupts a protein coding sequence resulting in inactivation of the respective gene product, e.g. ICP34.5. The marker may be one of the markers described above. The cassette may further comprise nucleic acid encoding a polyadenylation sequence, e.g. the Simian Virus 40 (SV40) polyadenylation sequence, located downstream (3′) of the nucleic acid encoding the marker.

The control sequence (or regulatory sequence), which may optionally include the carbohydrate response element, is preferably operably linked to the nucleic acid encoding insulin. The term “operably linked” may include the situation where a selected nucleotide sequence and regulatory nucleotide sequence are covalently linked in such a way as to place the expression of a nucleotide coding sequence under the influence or control of the regulatory sequence. Thus a regulatory sequence is operably linked to a selected nucleotide sequence if the regulatory sequence is capable of effecting transcription of a nucleotide coding sequence which forms part or all of the selected nucleotide sequence. Where appropriate, the resulting transcript may then be translated into a desired protein or polypeptide.

The exogenous insulin sequence (or proinsulin or preproinsulin sequence) may have been modified to contain one or more mutations compared to the wild type sequence. For example, His 10 of the B-chain of human insulin may have been modified to Asp in order to improve biopotency of the resulting mutant insulin compared to the wild type. Modifications may be introduced by recombinant DNA techniques and may comprise insertion, addition, substitution or deletion.

As described above, in other preferred arrangements the nucleic acid sequence encoding insulin may be modified such that one or more selected peptide motifs or predetermined sequences are incorporated in the amino acid sequence of one or more of preproinsulin, proinsulin or insulin. Such motifs may provide a specific site at which a selected protease may cleave the polypeptide chain.

In one preferred arrangement the amino acid sequence incorporates one or more of the amino acid motifs RTRR and/or RQKR. These sites represent specific sites at which the ubiquitous furin protease may cleave the polypeptide. Furin protease is present in a wide variety of cells. Accordingly, by positioning the peptide motifs at a site at which the polypeptide is naturally cleaved into the A, B and/or C chains it is possible to provide a means of post-translationally processing, in virtually any cell, any one or more of preproinsulin or proinsulin, e.g. by cleavage of the C-peptide. In this way cells that do not normally produce insulin may be infected with HSV according to the invention such that they become capable of producing insulin.

Ubiquitous proteases, e.g. those local to the endoplasmic reticulum, may cleave the leader signal sequence to generate proinsulin from preproinsulin. The proinsulin thereby produced may then be naturally processed to provide mature and active insulin by furin mediated cleavage of the C-peptide. The insulin thereby produced may then preferably be secreted from the cell.

This can be used to alleviate the need to specifically infect cells that have the natural cellular components capable of processing preproinsulin and/or proinsulin to provide mature and active insulin.

Of course, proteases other than furin may be selected. As such, the insulin encoded by the HSV may be a mutant that has been modified to encode a desired cleavage site for a selected protease, preferably a protease that occurs in the cells of the patient that are to be infected by the HSV upon administration. These cells may be those to which the HSV is directly administered or which the HSV infects following that administration.

The exogenous nucleic acid sequence may be of any size, e.g. up to 5, 10, 15, 20, 25, 30, 35, 40 or 45 Kbp, but is preferably up to 50 Kbp in length.

An administration step may comprise parenteral administration. Preferably such administration is by injection. The injection may be intravenous or intramuscular or be injection to the pancreas. Alternatively, administration may be oral or nasal.

A said disease may be a form of diabetes mellitus, preferably insulin dependent, Type I, diabetes mellitus. Alternatively, a said disease may be any other disease state involving an abnormal pattern of insulin expression or function and may be diabetes of any form e.g. early onset, mature onset or monogenic diabetes of the young (MODY).

The herpes simplex virus may be provided as a medicament, pharmaceutical composition or vaccine in combination with a pharmaceutically acceptable carrier, adjuvant or diluent. The composition may be formulated for topical, parenteral, intravenous, intramuscular, intrathecal, intraocular, subcutaneous, oral, inhalational or transdermal routes of administration which may include injection. Injectable formulations may comprise the selected compound in a sterile or isotonic medium.

The patient to be treated may be any animal or human. The patient may preferably be a non-human mammal, but is more preferably a human patient. The patient may be male or female.

Herpes simplex viruses of the invention may be used in ‘gene therapy’ or ‘gene delivery’ techniques in vitro or in vivo. Non-neurovirulent herpes simplex viruses of the invention are expression vectors and may be used to infect selected cells or tissues in order to express, and preferably secrete, a selected protein, preferably an exogenous protein selected from one or more of preproinsulin, proinsulin or insulin.

In one arrangement, cells may be taken from a patient, a donor or from any other source, infected with a herpes simplex virus of the invention, optionally screened for expression and/or secretion and/or function of the exogenous protein, and returned/introduced to the patient's body, e.g. by injection. This method may be used to treat a disease state, which may be a disease state associated with abnormal expression or function of the selected protein.

Delivery of herpes simplex viruses of the invention to the selected cells may be performed using naked virus or by encapsulation of the virus in a carrier, e.g. nanoparticles, liposomes or other vesicles.

In vitro cultured cells, preferably human or mammalian cells, transformed with viruses of the present invention and preferably cells expressing or secreting the selected protein as well as methods of transforming such cells in vitro with said viruses form further aspects of the present invention.

Insulin

In this specification, an insulin nucleic acid (which may also refer to a preproinsulin or proinsulin nucleic acid) may be any nucleic acid (DNA or RNA) having a nucleotide sequence having a specified degree of sequence identity to one of SEQ ID No.s 2 or 5, to an RNA transcript of any one of these sequences or to the complementary sequence of any one of these sequences. Alternatively an insulin nucleic acid may be one that hybridises to one of these sequences under high or very high stringency conditions. The specified degree of sequence identity may be from at least 60% to 100% sequence identity. More preferably, the specified degree of sequence identity may be one of at least 65%, 70%, 75%, 80%, 85%, 86%, 87%, 88%, 89%, 90%, 91%, 92%, 93%, 94%, 95%, 96%, 97%, 98% or 99% identity.

In this specification, an insulin polypeptide or protein (which may also refer to a preproinsulin or proinsulin polypeptide or protein) may be any peptide, polypeptide or protein having an amino acid sequence having a specified degree of sequence identity to one of SEQ ID No.s 1 or 6. The specified degree of sequence identity may be from at least 60% to 100% sequence identity. More preferably, the specified degree of sequence identity may be one of at least 65%, 70%, 75%, 80%, 85%, 86%, 87%, 88%, 89%, 90%, 91%, 92%, 93%, 94%, 95%, 96%, 97%, 98% or 99% identity.

The present invention is not limited to human insulin, human insulin derivatives or mutant forms of human insulin. The invention may extend to any other insulin or mutant form of insulin. For example, the insulin may be that of any animal, more preferably of any mammal or non-human mammal, e.g. rabbit, guinea pig, rat, mouse or other rodent (including any animal in the order Rodentia), cat, dog, pig, sheep, goat, cattle, horse or non-human primate.

Sequence Identity

Percentage (%) sequence identity is defined as the percentage of amino acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 amino acids and the candidate sequence comprises 10 amino acids, the candidate sequence can only have a maximum identity of 10% to the entire length of the given sequence. This is further illustrated in the following example:

(A)

Given seq: XXXXXXXXXXXXXXX (15 amino acids)

Comparison seq: XXXXXYYYYYYY (12 amino acids)

The given sequence may, for example, be that encoding insulin (e.g. SEQ ID No.1).

% sequence identity=the number of identically matching amino acid residues after alignment divided by the total number of amino acid residues in the longer given sequence, i.e. (5 divided by 15)×100=33.3%

Where the comparison sequence is longer than the given sequence, sequence identity may be determined over the entire length of the given sequence. For example:

(B)

Given seq: XXXXXXXXXX (10 amino acids)

Comparison seq: XXXXXYYYYYYZZYZZZZZZ (20 amino acids)

Again, the given sequence may, for example, be that encoding insulin (e.g. SEQ ID No.1).

% sequence identity=number of identical amino acids after alignment divided by total number of amino acid residues in the given sequence, i.e. (5 divided by 10)×100=50%.

Alignment for purposes of determining percent amino acid sequence identity can be achieved in various ways known to a person of skill in the art, for instance, using publicly available computer software such as BLAST, BLAST-2, ALIGN or Megalign (DNASTAR) software.

Identity of nucleic acid sequences may be determined in a similar manner involving aligning the sequences and introducing gaps if necessary, to achieve the maximum sequence identity, and calculating sequence identity over the entire length of the respective sequences. Where the aligned sequences are of different length, sequence identity may be determined as described above and illustrated in examples (A) and (B).

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1. Overview of construction of plasmid RL1.dCMV-hppI1.

(1) Excision of the cytomegalovirus (CMV) promoter (pCMV)-preproinsulin fragment from plasmid phppil by restriction endonuclease digestion with SspI and ECORV restriction enzymes.

(2) Restriction endonuclease digestion of plasmid RL1.del with the HpaI restriction enzyme followed by treatment with Calf Intestinal Phosphatase (CIP).

(3) Insertion of the pCMV-preproinsulin fragment obtained in (1) into the digested RL1.del of (2) and blunt end ligation to form plasmid RL1.dCMV-hppI1.

FIG. 2. Excision of the pCMV-preproinsulin fragment from plasmid phppI1.

Restriction enzyme digestion of plasmid phppI1 was followed by agarose gel electrophoresis to separate the restriction fragments. The approx. 1.25 Kbp bands were cut from the gel and purified by further agarose gel electrophoretic separation.

FIG. 3. Size confirmation of plasmid RL1.del.

Following HpaI digestion, agarose gel electrophoresis was used to confirm the apparent molecular weight of the linearised RL1.del.

FIG. 4. Determination of insertion of preproinsulin DNA in plasmid RL1.dCMV-hppI1.

EcoRI restriction enzyme digestion of ligated plasmid RL1.dCMV-hppI1 containing the pCMV-preproinsulin fragment was performed to confirm insertion of the pCMV-preproinsulin fragment and direction of insertion in individual clones.

FIG. 5. Determination of insertion of preproinsulin DNA in plasmid RL1.dCMV-hppI1.

Further confirmation of the insertion of the pCMV-preproinsulin fragment, and direction of insertion, in individual clones was performed by HindIII/XhoI restriction endonuclease digestion and agarose gel electrophoresis.

FIG. 6.

(A) Transfection of HSV-1 DNA with plasmid RL1.dCMV-hppI1 to generate HSV-1 expressing preproinsulin.

HSV-1 containing nucleic acid encoding the Green Fluorescent Protein (GFP) in a disabling locus (RL1), and normally expressing GFP, were co-transfected with plasmid RL1.dCMV-hppI1. Transfected virus was purified and selection made for virus not expressing GFP, but expressing preproinsulin. A high concentrate stock of transformed virus expressing preproinsulin was prepared.

(B) and (C) Production of HSV1716gfp/ppi.

(B) An identical cloning procedure was used to generate the plasmid RL1.dCMV-hppI-GFP except that the SspI/EcoRV fragment containing CMV-hppI was cloned into the BglII site of the plasmid R11.dIRES-GFP such that the CMV promoter will drive expression of the bicistronic mRNA encoding human preproinsulin in tandem with GFP.

(C) The linearized plasmid RL1.dCMV-hppI-GFP was co-transfected into BHK cells along with HSV-1 strain 17+ DNA and GFP-expressing plaques were selected.

FIG. 7. Results of proinsulin ELISA assay.

Key to ELISA wells:

A1, B1, C1—whole cell extract (wce) from Vero cells infected with HSV1716gfp

D1, E1, F1—wce from Vero cells infected with HSV1716gfp/ppi

G1, H1, A2—wce from BHK cells infected with HSV1716gfp

B2, C2, D2—wce from BHK cells infected from HSV1716gfp/ppi

E2, F2, G2—wce from C8161 cells infected with HSV1716gfp

H2, A3, B3—wce from C8161 cells infected with HSV1716gfp/ppi

C3, D3, E3—wce from HeLa cells infected with HSV1716gfp

F3, G3, H3—wce from HeLa cells infected with HSV1716gfp/ppi

A4, B4, C4—medium from Vero cells infected with HSV1716gfp

D4, E4, F4—medium from Vero cells infected with HSV1716gfp/ppi

G4, H4, A5—medium from BHK cells infected with HSV1716gfp

B5, C5, D5—medium from BHK cells infected with HSV1716gfp/ppi

E5, F5, G5—medium from C8161 cells infected with HSV1716gfp

H5, A6, B6—medium from C8161 cells infected with HSV1716gfp/ppi

C6, D6, E6—medium from HeLa cells infected with HSV1716gfp

F6, G6, H6—medium from HeLa cells infected with HSV1716gfp/ppi

A7, B7, E7, F7, G7, H7, C8, D8—medium from CHO cells infected with HSV1716gfp

C7, D7—medium from CHO cells infected with HSV1716gfp/ppi at 8 hrs

A8, B8—medium from CHO cells infected with HSV1716gfp/ppi at 24 hrs.

E8 kit control 1 proinsulin concentration 0

F8 kit control 2 proinsulin concentration 5 μmol/litre

G8 kit control 3 proinsulin concentration 15 μmol/litre

H8 kit control 3 proinsulin concentration 50 μmol/litre

FIG. 8. Proinsulin ELISA results showing colour formation for cell extracts and culture medium in BHK, Vero, CHO and C8161 cell cultures.

First two columns—HSV1716gfp/ppi infected cell cultures; Third column—HSV1716gfp infected cell cultures (control); fourth column wells 1-5: standard wells (wells 1-5: 0, 1.25, 13.7, 41.3 and 78.7 μmol/l proinsulin respectively); fourth column wells 6-8: duplicate samples as for column 1 wells 5-7 but at double concentration, indicated by x2, (well 6: BHK cell extract x2; well 7: Vero cell extract x2; well 8: CHO cell extract x2).

FIG. 9. C peptide ELISA (DakoCytomation™) results showing colour formation for cell extracts and culture medium in BHK, Vero, CHO and C8161 cell cultures.

First three columns—HSV1716gfp/ppi infected cell cultures; fourth column HSV1716gfp infected cell cultures (control); fifth column—standard wells (wells 1-5:0, 0.35, 3.2, 9.1 and 14.8 ng/ml C peptide respectively; wells 6-8: 78.7, 41.3 and 13.7 pmol/l proinsulin respectively).

FIG. 10. C peptide ELISA (IBL™) results showing colour formation for cell extracts and culture medium in BHK, Vero, CHO and C8161 cell cultures.

First three columns—HSV1716gfp/ppi infected cell cultures; fourth column HSV1716gfp infected cell cultures (control); fifth column—standard wells (wells 1-6: 0, 0.2, 0.7, 2.0, 6.0 and 16.0 ng/ml C peptide respectively; wells 7 and 8: 78.7 and 0 μmol/l proinsulin).

FIG. 11. Extract from accession no. NM_{—}000207 in the NCBI database (http://www.ncbi.nlm.nih.gov/) showing the amino acid sequence (SEQ ID No.1) and nucleotide sequence (SEQ ID No.2) of human proinsulin.

FIG. 12.

(A) Mutagenic primers were used to generate a mutant proinsulin. Each mutagenic primer (SEQ ID No.3 and SEQ ID No.4) had a single base substitution (underscored in SEQ ID No.s 3 and 4 in the figure). Base alterations from wild-type were confirmed by sequencing. The amino acid and nucleotide sequence of the mutant human proinsulin (HisB10Asp Proinsulin) is shown (SEQ ID No.s 5 and 6 respectively). The HisB10Asp mutation is underscored in the amino acid sequence. HisB10Asp Proinsulin also contains two tetrabasic motifs, RTRR and RQKR (italicised in the amino acid sequence) providing sites for furin cleavage. These represent mutation of L to R and K to R compared to the respective tetrapeptide motifs contained in the wild-type amino acid sequence (SEQ ID No.1; FIG. 11).

(B) Nucleotide sequence of the carbohydrate response element (SEQ ID No.7).

FIG. 13. Diagrammatic representation of plasmid His 10 Mutated pMTChINS having a size of 7817 Kb. The human insulin (hINS) cassette was mutated at the furin cleavage sites to create the amino acid sequence RTRR and RQKR. The plasmid includes a third mutation which creates an Aspartate 10 instead of His 10 at the B-chain. Mutagenesis of pMTChINS used 3 sets of primers. Unique sites in the plasmid are NotI and KpnI.

FIG. 14. Green fluorescent protein (GFP) expression in pig pancreatic islet cells (A), mouse pituitary AtT20 tumour cells (B), rat pancreatic islet cells (C) and NF107 human primary fibroblasts (D) after 16 hours of infection with 5pfu/cell HSV1716gfp/ppi.

FIG. 15.

(A) Linear representations of plasmids used in preparation of HSV1716MTC-ins.

(B) Representations of the genome structure around the RL1 locus of HSV1716MTC-ins and HSV1716gfp/ppi.

### DETAILED DESCRIPTION OF THE BEST MODE OF THE INVENTION

Specific details of the best mode contemplated by the inventors for carrying out the invention are set forth below, by way of example. It will be apparent to one skilled in the art that the present invention may be practiced without limitation to these specific details.

HSV1716 is a specific variant of HSV-1 strain 17 which is non-neurovirulent. It contains a 759 bp deletion in the Bam HI s restriction fragment located in each of the terminal and internal repeats (TR_{L} and IR_{L}—map units 0-0.02 and 0.81-0.83 respectively) and has been deposited under the Budapest Treaty at the European Collection of Animal Cell Cultures (ECACC), Porton Down, Salisbury, Wiltshire, United Kingdom under accession number V92012803. In this specification, reference to HSV1716gfp or HSV1716gfp/ppi is a reference to an HSV having the characteristics of non-neurovirulence of HSV1716 and may contain a modification in one or both of the long repeat regions (R_{L}) of the herpes simplex virus genome but is not necessarily identical to or derived from the deposited HSV1716 virus.

Materials and Methods

Plasmid phppI1

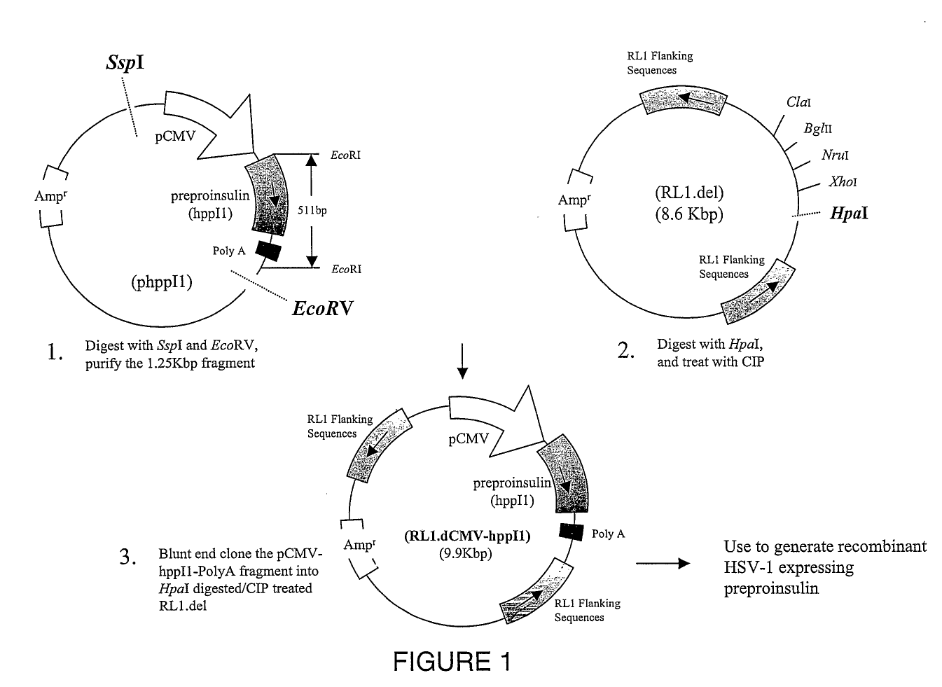

This plasmid was a kind gift from Prof. Kevin Docherty, University of Aberdeen, United Kingdom. The arrangement of sequence components in the plasmid is shown in FIG. 1.

Plasmid RL1.del

RL1.del was provided by Dr. E. McKie and is the pGEM-3Zf(−) plasmid (Promega) into which has been cloned an HSV-1 strain 17 fragment (123459-129403) consisting of the RL1 gene (ICP34.5) and its flanking sequences. The 477 bp PflMI-BstEII fragment of the RL1 gene (125292-125769) has been removed and replaced with a multi-cloning site (MCS) to form RL1.del.

Construction of Plasmid RL1.dCMV-hppI1

The general cloning strategy used is outlined in FIG. 1. A pCMV-preproinsulin fragment was excised from plasmid phppI1 and inserted in plasmid RL1.del between the two RL1 flanking sequences to create plasmid RL1.dCMV-hppI1.

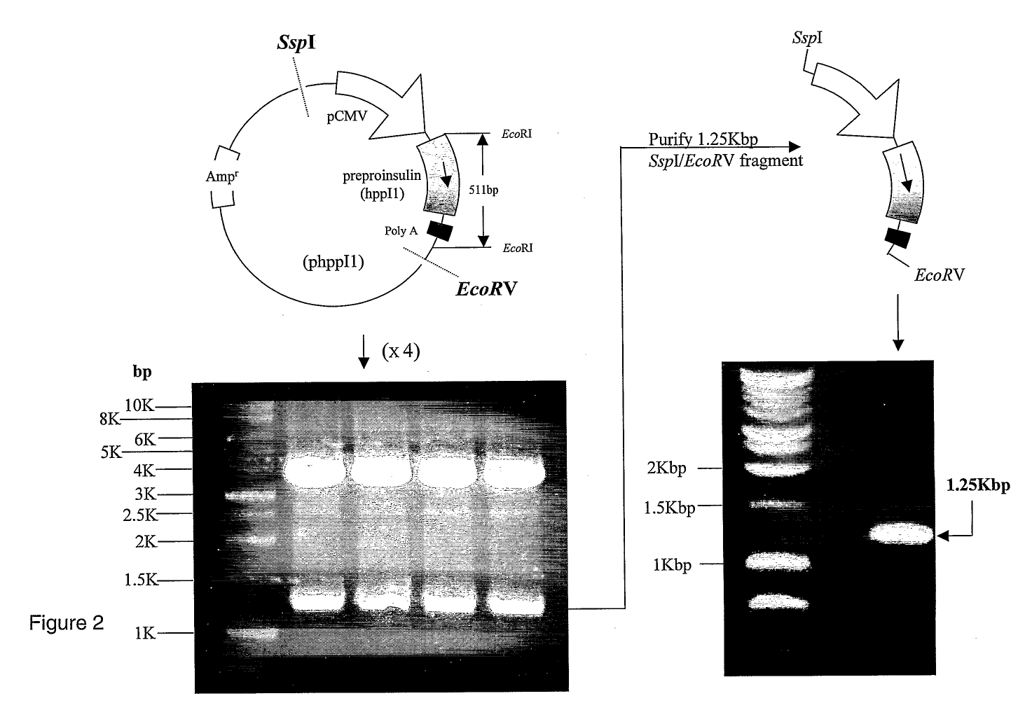

The fragment pCMV-preproinsulin comprising the CMV promoter and human preproinsulin coding sequence was excised from plasmid phppI1 by digestion with the SspI and EcoRV restriction enzymes. The products of the restriction digest were separated by agarose gel electrophoresis and the approx. 1.25 Kbp pCMV-preproinsulin was further purified by agarose gel electrophoresis (FIG. 2).

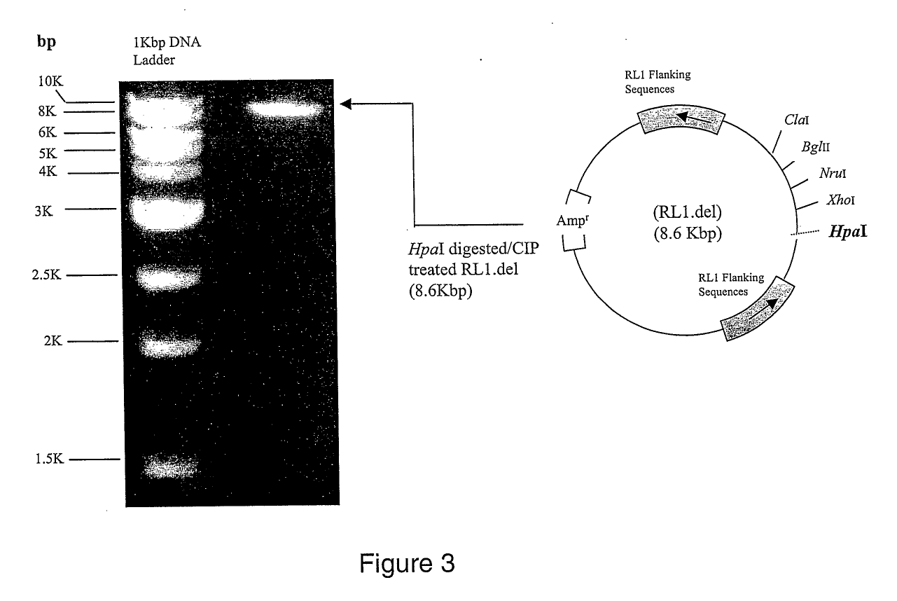

The plasmid RL1.del was subject to HpaI digestion to create an insertion site within the MCS. The digested plasmid was further subjected to treatment with Calf Intestinal Phosphatase (CIP) to create blunt ends at the insertion site and subjected to agarose gel electrophoresis to confirm an approximate size of 8.6 Kbp (FIG. 3).

The pCMV-preproinsulin fragment and digested RL1.del was then blunt-end ligated using T4 DNA Ligase to generate plasmid RL1.dCMV-hppI1 (FIG. 1).

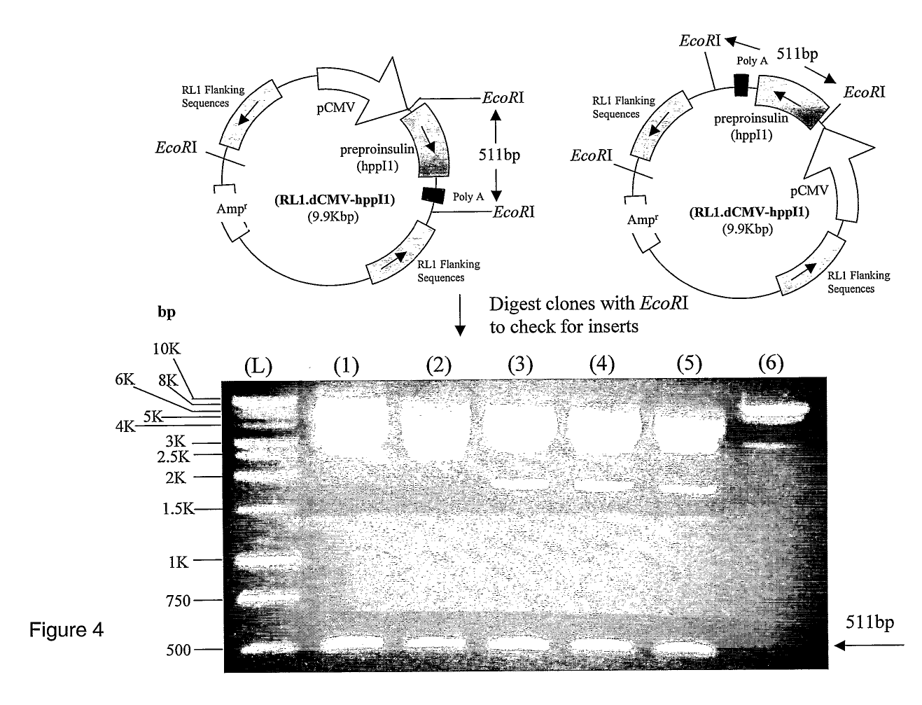

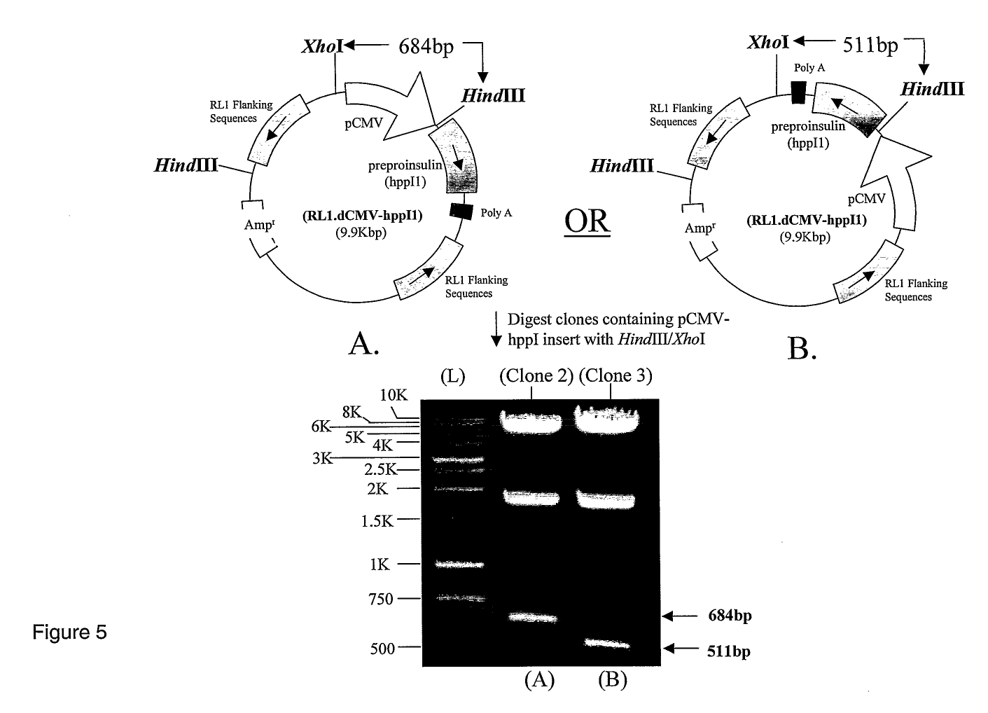

The recombinant clones were subjected separately to EcoRI and HindIII/XhoI digestion (FIGS. 4 and 5 respectively). In both cases the products of plasmid digestion were separated by agarose gel electrophoresis in order to confirm insertion of the PCMV-preproinsulin fragment and the direction of insertion. The presence of a 51 lbp preproinsulin-polyA sequence fragment following EcoRI digestion (FIG. 4) and the presence of a 684 bp pCMV fragment and 51 lbp preproinsulin-polyA sequence fragment following HindIII/XhoI digestion (FIG. 5) confirmed insertion of the pCMV-preproinsulin fragment into RL1.del.

Generation of Herpes Simplex Virus Expressing Preproinsulin

Plasmid RL1.dCMV-hppI1 was linearised by digestion with the restriction endonuclease ScaI and purified by agarose gel electrophoresis to confirm an apparent molecular weight of 9.9 Kbp.

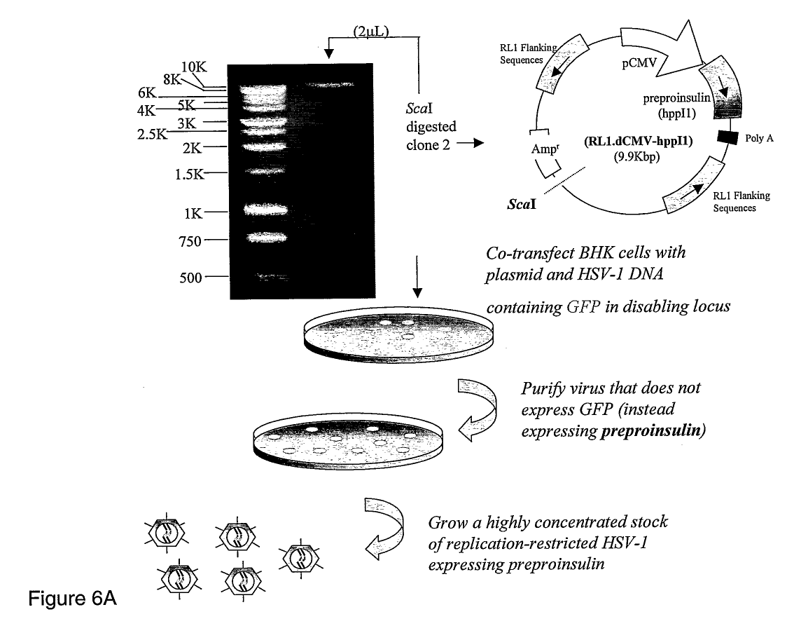

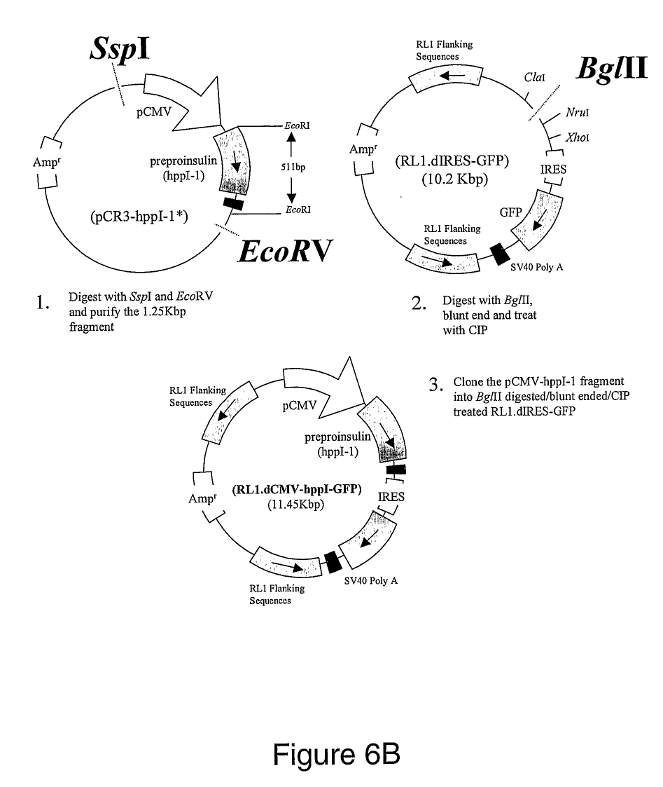

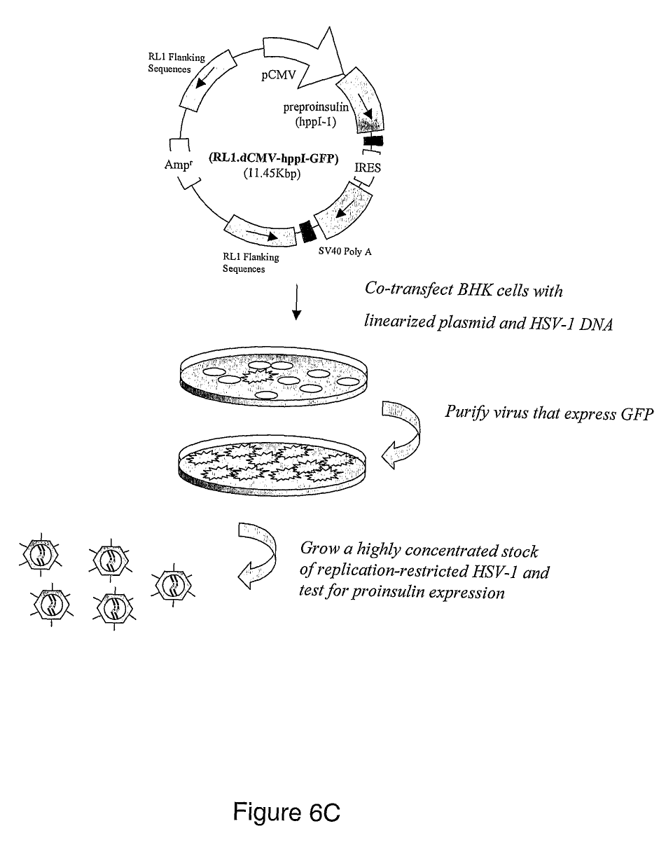

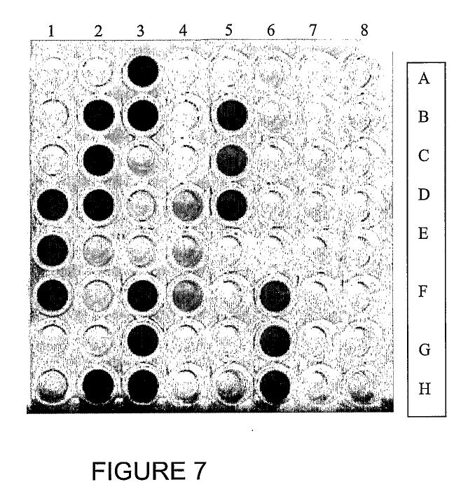

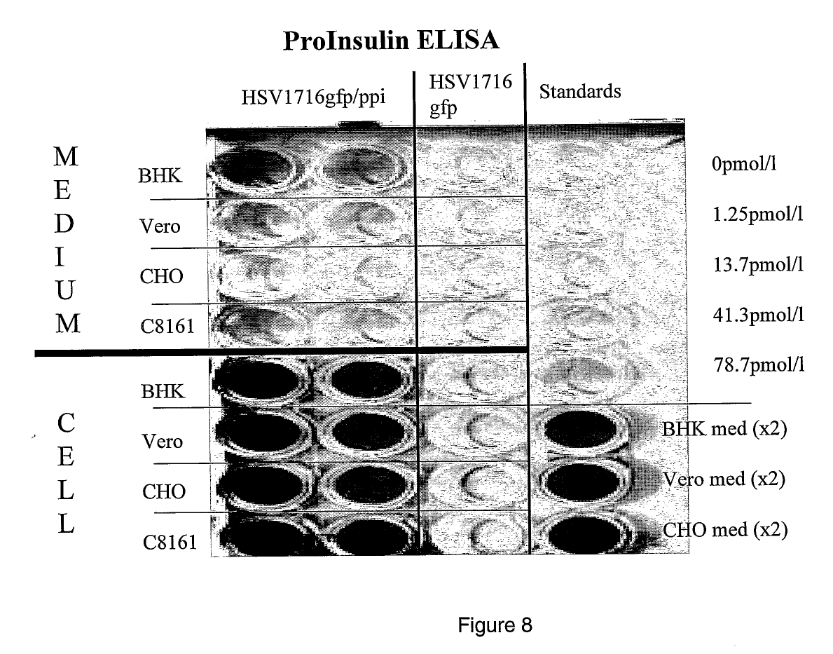

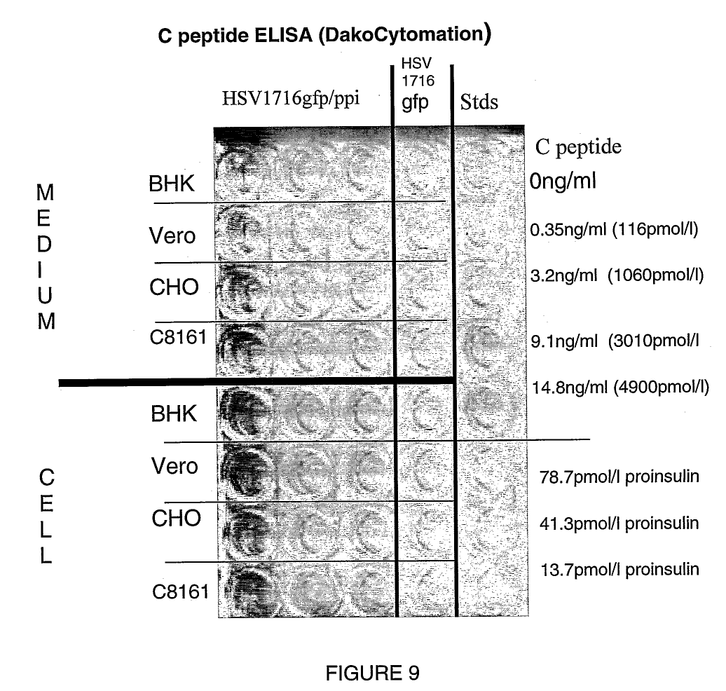

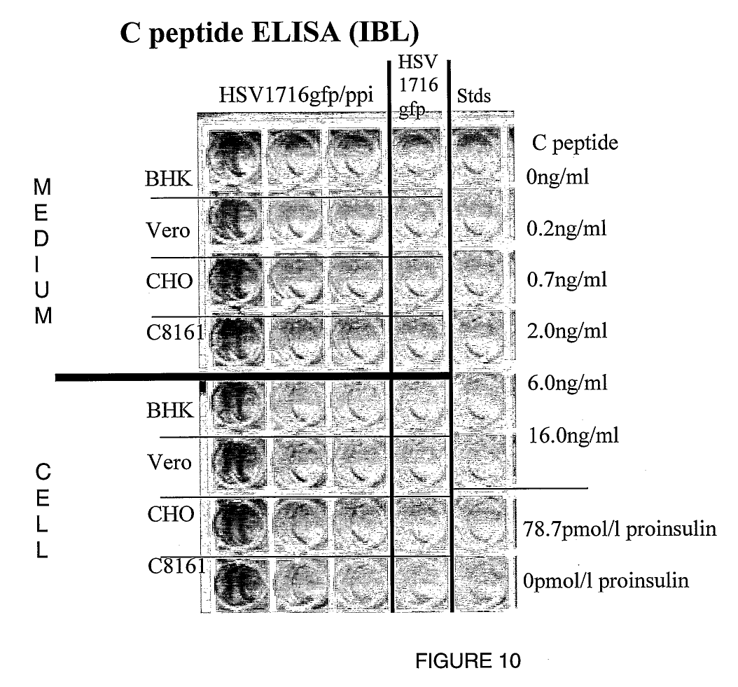

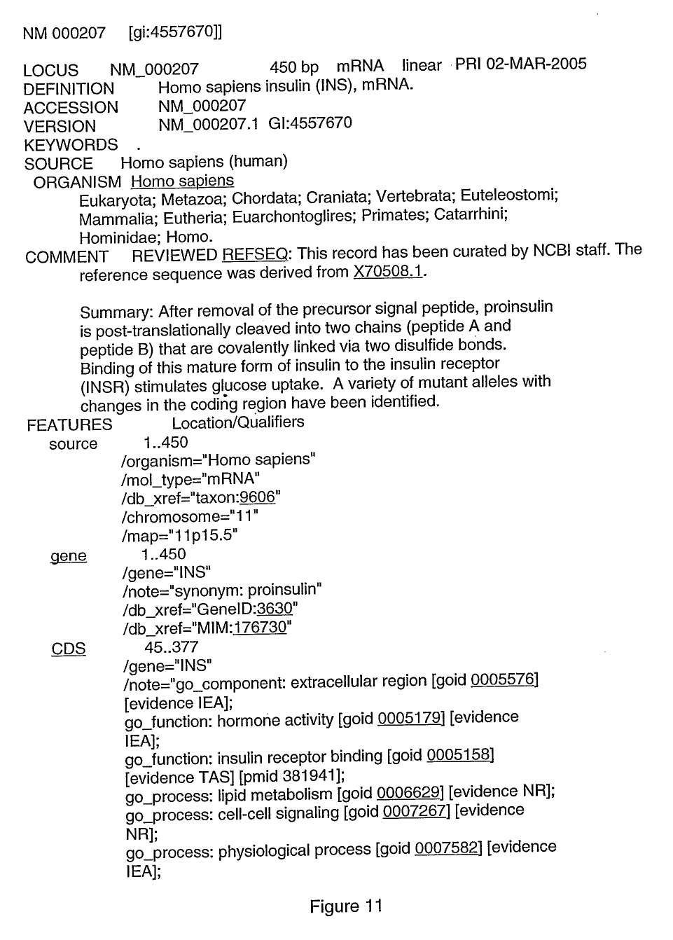

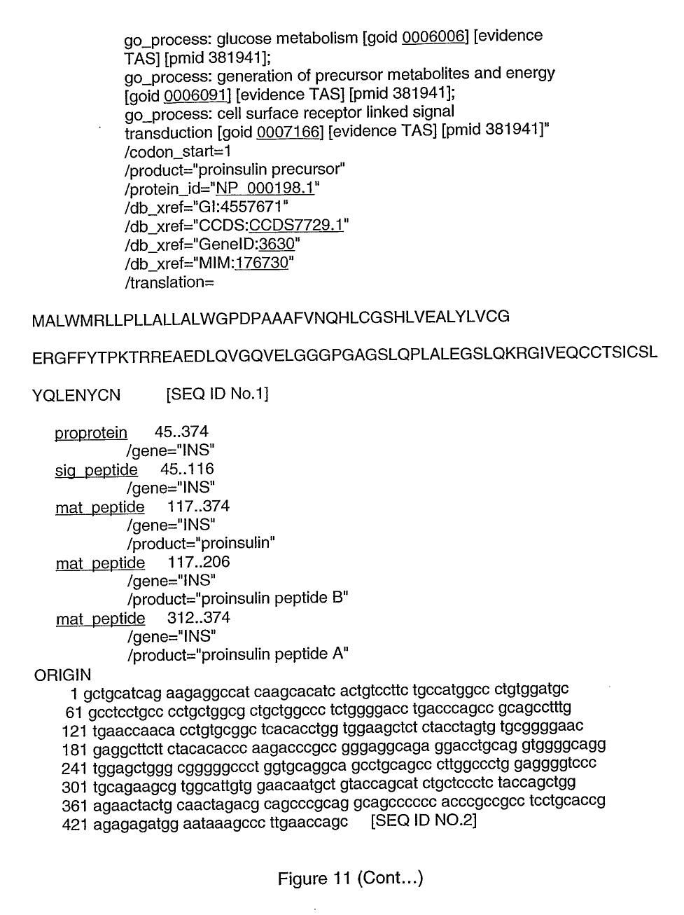

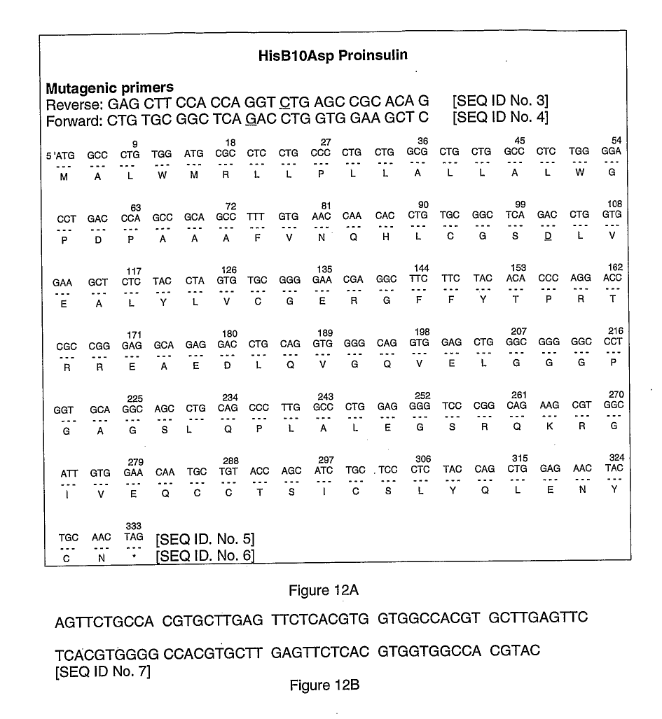

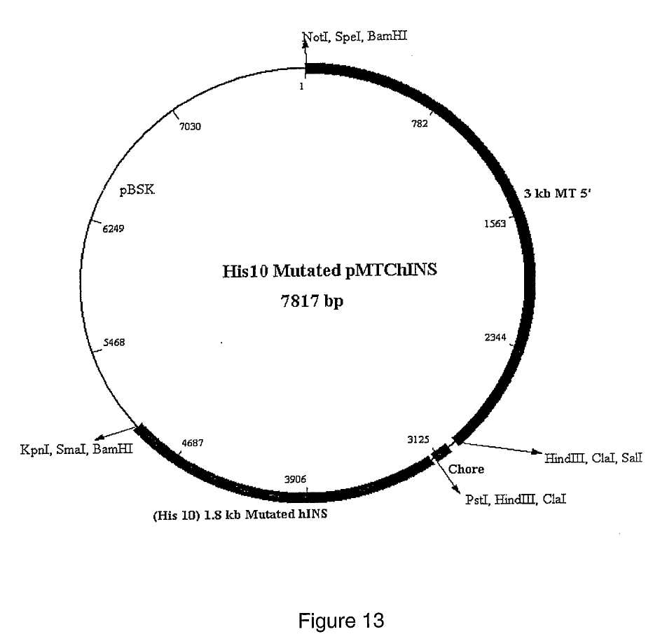

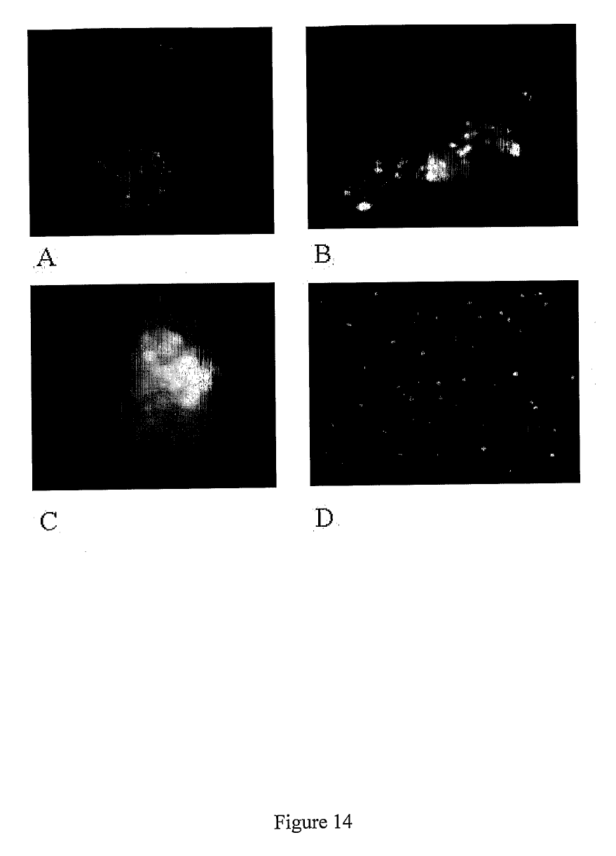

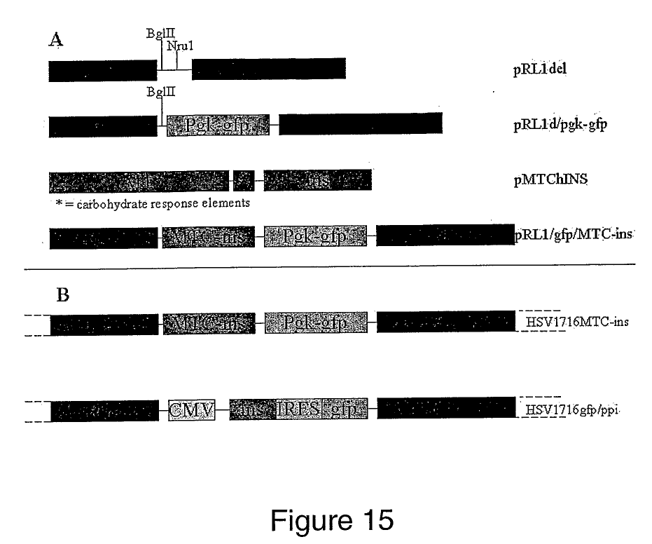

BHK cells were then co-transfected with linearised RL1.dCMV-hppI1 plasmid DNA and HSV-1 DNA having the GFP coding sequence inserted in the RL1 locus and expressing GFP (HSV1716gfp). A screen was performed to select for transformed virus by lack of GFP expression owing to homologous recombination between the RL1 flanking sequences of plasmid RL1.dCMV-hppI1 and the HSV-1 genomic RL1 sequences. The homologous recombination resulting in insertion of preproinsulin and lack of functional GFP expression (FIG. 6A).

An additional virus was generated such that preproinsulin was expressed in tandem with GFP using the plasmid RL1.dIRES-GFP. An identical cloning procedure was used to generate the plasmid RL1.dCMV-hppI-GFP except that the SspI/EcoRV fragment containing CMV-hppI was cloned into the BglII site of the plasmid R11.dIRES-GFP such that the CMV promoter will drive expression of a bicistronic mRNA encoding human preproinsulin in tandem with GFP. The linearized plasmid RL1.dCMV-hppI-GFP was co-transfected into BHK cells along with HSV-1 strain 17+ DNA and GFP-expressing plaques were selected (FIGS. 6B and 6C).

Transformed plaques were picked and used to form a high concentrate stock of HSV expressing preproinsulin. The transformed virus was called HSV1716gfp/ppi and was used in the following expression studies.

Expression Assays

Prior to infection, cells were plated out on 35 mm plates in 1 ml of a modified Eagle's medium containing 10% calf serum and incubated overnight. A range of cell types was used including Vero, BHK, C8161 (a human melanoma cell line) HeLa and CHO cells and each cell type was infected in triplicate with either 5 pfu/cell HSV1716gfp (containing the green fluorescent protein gene inserted at the RL1 locus) or HSV1716gfp/ppi (further containing the human preproinsulin gene inserted at the RL1 locus thereby inactivating the expression of functional ICP34.5).

Vero, BHK and C8161 cells are fully permissive for HSV infection, CHO cells are infected at very low levels (5-10% of cells).

Following infection, cells were incubated for 8 hrs and the medium removed and stored at −20° C. Cells were harvested by scraping into 1 ml PBS and, after pelleting by centrifugation, the cells were re-suspended in 100 μl PBS with 0.1% Tween 20 and stored at −20° C. Cells were thawed the following morning, vortexed and incubated on ice for 20 mins. Cell debris was removed by centrifugation to give a whole cell extract (wce) for ELISA. Media and wce were analysed for the presence of insulin using the DakoCytomation Intact™ Proinsulin ELISA (Cat No K6247) and for C peptide using the DakoCytomation™ C peptide ELISA (Cat No K6218) and the IBL C peptide ELISA (Cat No RE53011). Procedures for each assay were carried out according to the manufacturer's instructions.

In a separate experiment duplicate 35 mm plates of CHO cells in 2 ml of medium were infected with either HSV1716gfp or HSV1716gfp/ppi at 5pfu/cell and medium for proinsulin ELISA harvested at 8 and 24 hrs post infection.

Values for proinsulin and C-peptide concentrations were derived from standard curves.

Results and Notes on ELISA Procedures

Proinsulin

Two separate monoclonal antibodies were used to detect proinsulin in accordance with the protocol prescribed in the DakoCytomation Intact™ Proinsulin ELISA kit.

Intracellular concentrations of proinsulin in all cell types tested were far higher than the highest control provided with the kit (78.7 pmol/litre) and hence could not be determined. Although concentrations detected in the medium are lower there is a dilution factor due to the larger volume of medium (0.2 ml for intracellular extract vs. 0.5 ml for medium).

Photographs of ELISA plates for the proinsulin assay are shown in FIGS. 7 and 8.

Colour developed rapidly in the wce wells from cells infected with HSV1716gfp/ppi such that the absorbance was maximal within a few minutes. The colour appeared much faster than in the high concentration controls provided with the kit (e.g. FIG. 7 well H8, OD 0.428 after 10 mins, proinsulin concentration 50 pmol/litre).

Readings for Vero cells infected with HSV1716gfp/ppi were 1.726, 1.703 and 1.720, for BHK readings were 1.379, 1.484 and 1.744, for C8161 readings were 1.228, 1.064 and 1.379 and for HeLa cells readings were 1.720, 1.553 and 1.233. All readings for wce from cells infected with HSV1716gfp were within the background range (less than 0.05).

Intact proinsulin was also detected in the media of all cells infected with HSV1716gfp/ppi (FIGS. 7 and 8). OD readings were lower than for their corresponding wce but were mostly several fold higher than the highest concentration control provided with the kit. For Vero cells readings were 0.845, 0.603 and 0.913, for BHK readings were 1.343, 1.308 and 1.553, for C8161 readings were 0.448, 0.281 and 0.303 and for HeLa cells readings were 1.550, 1.476 and 1.155. For the media from cells infected with HSV1716gfp all readings were less than 0.03.

Intact proinsulin was also detected at low levels in the media from CHO cells infected with HSV1716/gfp/ppi at 8 hrs (OD readings of 0.172 and 0.226) and at 24 hrs (OD readings of 0.204 and 0.224). Readings for media from CHO cells infected with HSV1716gfp were all less than 0.025.

Approximate concentrations of proinsulin in the medium for HSV1716gfp/ppi infected cells are shown in Table 1.

TABLE 1

| Cell type | Concentration of Proinsulin in medium (pmol/l)) |
|---|---|
| BHK | >78.7 |
| Vero | 60 |
| CHO | 5 |
| C8161 | 50 |

Lower CHO levels are consistent with the poor HSV permissive properties of these cells. Levels for cells and medium from infection with HSV1716gfp were all at background.

C Peptide (DakoCytomation™)

Two monoclonal antibodies are used to detect the C peptide in accordance with the protocol prescribed in the DakoCytomation™ C peptide ELISA kit.

Values obtained for intracellular amounts of C peptide following infection with HSV1716gfp/ppi ranged from 6-15 ng/ml and for medium the range was 0.3-1.5 ng/ml.

C peptide values for cells and medium from HSV1716gfp infection were all below the detection limit of the assay.

A 63% cross reactivity has been reported in this assay with intact human proinsulin (biosynthetic) and between 71-87% cross reactivity with various, partially processed proinsulins. However, in the assay performed there was very little cross reactivity with the highest proinsulin standard (100 pmol/litre) provided with the proinsulin kit.

Optical density (OD) readings for medium and cell extracts from HSV1716gfp infection were mostly within the background range although some faint colour was visible in Vero and C8161 wells (FIG. 9). This is most probably carry over from the adjacent very positive wells. Some faint colour was also detected in the wells to which human proinsulin was added indicating low levels of assay cross-reactivity.

Sample quantities of C peptide found in the HSV1716gfp/ppi infected cells and culture media are shown in Table 2.

TABLE 2

| | Medium (ng/ml) | Cell (ng/ml) |
|---|---|---|
| BHK | 1.5 | 14.8 |
| Vero | 0.9 | 14.8 |
| CHO | 0.3 | 6.0 |
| C8161 | 1.2 | >14.8 |

C Peptide (IBL™)

Competition for antibody binding between a fixed amount of labelled C peptide and any unlabelled C peptide present in the sample forms the basis of this ELISA.

Optical density readings are inversely proportional to the concentration of C peptide in the unknown sample. High levels of unlabelled C peptide in a sample will compete out the binding of labelled C peptide to the coating antibody with subsequent low levels of colour development. Conversely, with low levels of C peptide in a sample there will be limited competition and high levels of colour development.

Sample quantities of C peptide found in HSV1716gfp/ppi infected cells and culture medium are shown in Table 3.

TABLE 3

| | Medium (ng/ml) | Cell (ng/ml) |
|---|---|---|
| BHK | 0.2 | 7.5 |
| Vero | 0.1 | 6.0 |
| CHO | 0.0 | 0.2 |
| C8161 | 0.2 | 7.0 |

Values obtained for intracellular amounts of C peptide following infection with HSV1716gfp/ppi ranged from 6-7.5 ng/ml and for medium the range was 0.1-0.2 ng/ml. C peptide values and OD readings for cell extracts and medium from HSV1716gfp infection were all below the detection limit of the assay, i.e. within the background range.

Values for C peptide are lower than those obtained for the same samples in the DakoCytomation C peptide ELISA (see above) most probably because of the cross reactivity in the latter assay with proinsulin. Thus the IBL C-peptide ELISA results suggest that some C peptide is derived from the bona fide processing of proinsulin in these cells.

This assay is reported to have 0.5% cross-reactivity with human proinsulin, a background reading was obtained for the well to which 78.7 pmol/l was added.

HSV176gfp/ppi—Further Expression Data

Both HSV1716gfp and HSV1716gfp/ppi were able to infect a variety of different primary cell types including rat and pig pancreatic islet cells, human primary fibroblasts (FIG. 14). Also shown in FIG. 14B is infection of the mouse pituitary tumour cell line AtT20 by HSV1716gfp. This cell line was used as it has been shown to have peptidase activity against preproinsulin and proinsulin and is capable of processing these forms of insulin to the mature protein.

Following infection of various cells with 5pfu/cell HSV1716gfp or HSV1716gfp/ppi, medium was removed at various time points and insulin and C-peptide levels were determined by radioimmunoassay (Table 4).

TABLE 4

| | Time post infection | | | | | |
|---|---|---|---|---|---|---|
| | 8 hours | | 24 hours | | 48 hours | |
| Virus/cell | C-peptide | Insulin | C-peptide | Insulin | C-peptide | Insulin |
| type | (ng/ml) | (ng/ml) | (ng/ml) | (ng/ml) | (ng/ml) | (ng/ml) |
| 1716gfp*/BHK | 130 | 6.2 | | | | |
| B16I**/BHK | 520 | 54 | | | | |
| 1716gfp/Hep2 | 120 | 5.6 | | | | |
| B16I/Hep2 | 350 | 38 | | | | |
| 1716gfp/AtT20 | <100 | <2 | | | 130 | 2 |
| B16I/AtT20 | 150 | 10 | | | 275 | 10 |
| 1716gfp/NF107 | .115 | 9.5 | 220 | 6.0 | 200 | 5.1 |
| B16I/NF107 | 425 | 32 | 300 | 8.7 | 300 | 9.7 |
| 1716gfp/pig islets | | | 270 | 2861 | 320 | 3636 |
| B16Igfp/pig islets | | | 360 | 3314 | 360 | 4169 |

*1716gfp = control virus (HSV1716gfp)
**B16I = HSV1716 expressing human preproinsulin (HSV1716gfp/ppi)
BHK = baby hamster kidney
AtT20 = mouse pituitary tumour
NF107 = human primary fibroblasts

For BHK and Hep2 approximately 2×10⁶ cells were infected at 5pfu/cell, for NF107 approximately 1×10⁶ cells were infected at 5pfu/cell. For AtT20 and pig islet cells, estimation of cell numbers is problematical as they grow in clusters but approximately 1×10⁶ cells were infected at 5pfu/cell.

As can be seen from Table 4 infection of cells that do not normally produce insulin (BHK, Hep2, AtT20 and NF107) with the HSV1716gfp control virus resulted in some apparent detection of C-peptide and/or insulin. However, the level of detection encountered was within the range of background readings and does not represent insulin production by these cells.

Porcine insulin is detected by the assay used. Accordingly, in pig islet cells where background porcine insulin levels are high, expression of human insulin resulting from HSV1716gfp/ppi could not be distinguished. However, an increase in C-peptide levels could be detected.

Owing to the cross reactivity between porcine and human insulin and the high level of insulin production by pancreatic islet cells most of the insulin levels from pig islet cells were outside the range of the standard curve even at a 1:40 dilution and these results are not shown. For these samples, the mean C-peptide concentrations for HSV1716gfp and B16I were 300 ng/ml and 435 ng/ml respectively, suggesting that HSV1716gfp/ppi infection augments insulin production in pig pancreatic islet cells. This was confirmed by results from one experiment where values were within the standard curve range and indicate a 20% boost to insulin production in pig pancreatic islet cells infected with HSV1716gfp/ppi. No cytopathic effects were observed by light microscopy in the pig pancreatic islet cells infected with either HSV1716gfp or HSV1716gfp/ppi suggesting that the cells were not lytically infected.

FIG. 14 shows green fluorescent protein expression in pig and rat pancreatic islet cells, mouse pituitary tumour cells and human primary fibroblasts after 16 hours of infection with 5 pfu/cell HSV1716gfp/ppi.

Expression of Insulin by HSV1716 with MTC-ins Insert

A mutant of HSV1716, MTC-ins, with a preproinsulin gene, whose expression is controlled by a promoter combining the human metallothionein promoter with carbohydrate response elements, was generated by insertion of the MTC-ins cassette into the RL1 locus of HSV-1 strain 17+.

The proinsulin DNA was genetically modified to incorporate the RTRR and RQKR tetrapeptide motifs such that the proinsulin expressed is cleavable by the ubiquitous furin protease and can therefore be processed by many different cell types (furin protease is endogenous to a wide variety of cells). In addition, the construct has a mutation in the B-chain, His10 to Asp-10, which is known to increase biopotency. The plasmid, pMTCChINS, for construction of this recombinant virus was kindly provided by Prof. Oi Lian Kon, National Cancer Centre, Singapore.

The MTC-ins expression cassette was cloned into the shuttle vector pRLld/pgk-gfp, which contains a green fluorescent protein (GFP) gene whose expression is driven by the phosphoglycerokinase (PGK) promoter, in both orientations relative to the PGK-gfp cassette. Plasmid RL1d/pgk-gfp was derived from pRL1del (FIG. 1) by insertion of the pgk-gfp expression cassette into the NruI site of pRL1del. pRL1del contains the sequence of nucleotides 123459-129403 of the RL1 locus of HSV-1 strain 17+ in which restriction sites have been incorporated enabling insertion of a selected nucleotide sequence in the RL1 locus. This plasmid can then be used to introduce, by homologous recombination of the flanking RL1 sequences, the selected sequence in the RL1 locus of HSV-1 strain 17+. The use of two independent promoters to force preproinsulin and GFP expression was required as the preproinsulin DNA (1.8 kbp) present in the MTC-ins plasmid incorporates the 3′ untranslated region of the human preproinsulin gene with associated polyadenylation signal and it was assumed that this would interfere with the IRES-gfp expression if the insert was cloned into RL1.dIRES-gfp. This was shown to be the case as the MTC-ins insert was cloned into RL1.dIRES-gfp but no gfp expression was detected either directly from the recombinant plasmid or from recombinant viruses generated by homologous recombination.

The MTC-ins expression cassette (4.8 kbp) was excised from pMTChINS by BamHI digestion and blunt ended and ligated into the BglII digested, blunt ended and CIAP-treated pRL1d/pgk-gfp. Linear representations of these plasmids are shown in FIG. 15A.

Recombinant plasmids were linearized by XmnI digestion and two recombinant viruses, HSV1716MTC-ins3 and HSV1716MTC-ins6, were derived by homologous recombination.

Southern analysis of the plaque-purified viruses confirmed that their genomes contained the 6 kbp MTC-ins/PGK-gfp cassettes in the RL1 locus and virus stocks were prepared.

Representations of the genome structure around the RL1 locus of HSV1716MTC-ins relative to the structure of HSV1716gfp/ppi are shown in FIG. 15B. Proinsulin expression in MTC-ins is independent of GFP expression as separate promoters are used whereas, in HSV1716gfp/ppi, insulin and gfp are expressed as a bicistronic mRNA using a CMV promoter and internal ribosome entry site (IRES).

To demonstrate expression of the proinsulin transgene, different cell types (Vero, BHK and HuH7 (a human hepatoma cell line)) were infected with either 5pfu/cell HSV1716MTC-ins or a control virus, HSV1716FVIII (which expresses murine Factor VIII).

Following infection, cells were incubated for 24 hrs and the medium removed. Cells were harvested by scraping into 1 ml PBS and, after pelleting by centrifugation, the cells were resuspended in 100 μl PBS with 0.1% Tween 20 and stored at −20° C. Cells were thawed the following morning, vortexed and incubated on ice for 20 mins. Cell debris was removed by centrifugation to give a whole cell extract (wce) for ELISA. Media and whole cell extract were analysed for the expression of insulin using the DakoCytomation Insulin ELISA (Cat No. K6219) and for C peptide using the DakoCytomation C peptide ELISA (Cat No. K6218). Values for C-peptide concentrations are derived from standard curves.

The Insulin ELISA has low cross reactivity with proinsulin and was chosen because the MTC-ins virus expresses proinsulin which is furin cleavable and it was assumed that this protease would be present in the cell types used. For the C-peptide ELISA, there is 63% cross reactivity with intact human proinsulin (biosynthetic) and between 71-87% cross reactivity with various, partially processed proinsulins.

Experiment 1. Infection of HuH7 and BHK cells with MTC-ins3, MTC-ins6, HSV1716gfp/ppi (B16I) or control viruses in the presence of 60 microM Zn²⁺.

TABLE 5

| | BHK | | | | HuH7 | | | |
|---|---|---|---|---|---|---|---|---|
| | Insulin | | C-peptide | | Insulin | | C-peptide | |
| | (pmol/l) | | (pmol/l) | | (pmol/1) | | (pmole/l) | |
| Virus | Medium | wce | Medium | wce | Medium | wce | Medium | wce |
| MTCins3 | 0 | 0 | 1200 | 100 | 0 | 0 | 300 | 100 |
| MTCins6 | 0 | 0 | 1000 | 120 | 0 | 0 | 500 | 100 |
| B16I | 15* | 25* | 400* | 115* | 5* | 50* | 550* | 500* |
| FVIII | 0 | 0 | 0 | 0 | 0 | 0 | 0 | 0 |
| Mock | 0 | 0 | 0 | 0 | 0 | 0 | 0 | 0 |

*Insulin/C-peptide values for HSV1716gfp/ppi (B16I; encoding non-furin cleavable proinsulin) should be treated with caution and are probably the result of cross reactivity of the detection kit with the proinsulin or partially processed proinsulin expressed by HSV1716gfp/ppi.

Comments on Experiment 1:

Despite the failure of the insulin ELISA to detect expression from MTC-ins, C-peptide results clearly demonstrate transgene expression and it seems most likely that the antibodies used in the insulin ELISA do not react with the genetically modified insulin expressed by the MTC-ins virus. The mutations introduced to allow furin cleavage are most likely responsible for the ablation of monoclonal antibody binding in the ELISA.

The orientation of the MTC-ins cassette does not appear to have a major effect on proinsulin expression.

Experiment 2. Infection of HuH7 cells with MTC-ins3, MTC-ins6, HSV1716gfp/ppi or control virus in the presence of 60 microM Zn²⁺ or 60 microM Zn²⁺/4.2 mM glucose.

TABLE 6

Insulin (pmol/litre)

| | Whole cell extract | | | medium | | |
|---|---|---|---|---|---|---|
| Virus | 1 | 2 | 3 | 1 | 2 | 3 |
| MTC- | 0 | 0 | 0 | 0 | 0 | 0 |
| ins3 | | | | | | |
| MTC- | 0 | 0 | 0 | 0 | 0 | 0 |
| ins6 | | | | | | |
| B16I | >10* | 15* | 12* | 25* | 45* | 33* |
| FVIII | 0 | 0 | 0 | 0 | 0 | 0 |

TABLE 7

| | C-peptide (pmol/litre) | | | | | |
|---|---|---|---|---|---|---|
| | Whole cell extract | | | Medium | | |
| Virus | 1 | 2 | 3 | 1 | 2 | 3 |
| MTC- | 120 | 350 | 500 | 180 | 300 | 1000 |
| ins3 | | | | | | |
| MTC- | 150 | 380 | 600 | 200 | 400 | 980 |
| ins6 | | | | | | |
| B16I | 160* | 200* | 250* | 500* | 400* | 600* |
| FVIII | 0 | 0 | 0 | 0 | 0 | 0 |

1 = normal culture medium
2 = normal culture medium + 60microM Zn²⁺
3 = normal culture medium + 60microM Zn²⁺/4.2 mM glucose
* Insulin/C-peptide values for HSV1716gfp/ppi (B16I; encoding non-furin cleavable proinsulin) should be treated with caution and are probably the result of cross reactivity of the detection kit with the proinsulin or partially processed proinsulin expressed by HSV1716gfp/ppi.

Comments on Experiment 2:

60 microM Zn²⁺ and 60 microM Zn²⁺/4.2 mM glucose clearly induces transgene expression from the MTC-ins virus with the highest levels being produced by 60 microM Zn²⁺/4.2 mM glucose.

60 microM Zn²⁺ and 60 microM Zn²⁺/4.2 mM glucose have little effect on transgene expression from HSV1716gfp/ppi (B16I).

Despite the failure of the insulin ELISA to detect expression from MTC-ins, C-peptide results clearly demonstrate transgene expression and it seems most likely that the antibodies used in the insulin ELISA do not react with the genetically modified insulin expressed by the MTC-ins virus.

The orientation of the MTC-ins cassette does not appear to have a major effect on proinsulin expression

Analysis of Vero cell medium infected with MTC-ins, which were negative for expression using the Insulin ELISA, with the C-peptide ELISA indicated transgene expression in these cells with 500 pmol/l C-peptide for both MTC-ins 3 and 6. Values for FVIII infected or mock infected Vero cells were 0.

### CONCLUSIONS

Following cellular infection with HSV1716gfp/ppi human proinsulin is expressed at high levels intracellularly and is secreted from the infected cells to the surrounding culture medium.

The amount of proinsulin produced following infection with HSV1716gfp/ppi is dependent on the level of HSV1716gfp/ppi infection as the poorly permissive CHO cells express lower levels than the fully permissive BHK, Vero and C8161 cells.

Cleaved C peptide was also detected both intracellularly and in the culture medium indicating that mature form insulin is present intracellularly and is secreted to the culture medium.

The results indicate that a non-neurovirulent herpes simplex virus incorporating an exogenous DNA sequence in the herpes simplex virus genome can be used to obtain safe and efficient expression of a protein encoded by the exogenous DNA sequence in a range of cell types, including mammalian and human cells. In particular, a non-neurovirulent herpes simplex virus expressing human or mammalian preproinsulin can be used to efficiently and stably express the preproinsulin polypeptide and obtain cleavage of the C-peptide fragment, i.e. post-translational processing of the preproinsulin to mature insulin and secretion of the mature insulin takes place.

Proinsulin is produced in large amounts following infection with HSV1716gfpppi and mature form insulin is readily secreted into the medium.

Incorporation of a carbohydrate response element and metallothionein promoter sequence operably linked in the HSV to the insulin nucleic acid has been used to demonstrate that expression of mature form insulin is inducible in a range of cell types following infection with the virus. This provides a means of regulating expression of the encoded insulin and a means by which the levels of transfected insulin in the patient may be controlled to meet the demands of the body.

By modifying the insulin nucleic acid sequence encoded by the HSV the inventors have demonstrated that it is possible to obtain processing of the encoded polypeptide by a protease endogenous to the infected cell. In particular, the inventors have taken advantage of the presence of the ubiquitous furin protease to demonstrate this. Incorporation of two tetrapeptide motifs at the site of chain cleavage by the natural insulin-specific proteases endogenous to insulin producing cells has enabled mature form insulin to be obtained in cells that do not normally produce insulin.

### REFERENCES

1. BL Liu, M Robinson, Z-Q Han, R H Branston, C English, Preay, Y McGrath, S K Thomas, M Thornton, P Bullock, C A Love and R S Coffin; Gene Therapy (2003) 10, 292-303.
2. WO 92/13943
3. A Dolan, E Mckie, A R Maclean, D J McGeoch; Journal of General Virology (1992) 73 971-973.
4. Aidan Dolan, Fiona E Jamieson, Charles Cunnigham, Barbara C Barnett Duncan J McGeoch; Journal of Virology March 1998 2010-2021.
5. Joany Chou, Earl R Kern, Richard J Whitley, Bernard Roizman; Science (1990) 250 1262-1265.
6. Coffin R S, MacLean A R, Latchman D S, Brown S M; gene therapy (1996) October 3(10) 886-91.
7. McKie E A, Hope R G, Brown S M, Maclean A R; Journal of General Virology, (1994) April 75(Pt4) 733-41.
8. McKay E M, McVey B, Marsden H S, Brown S M, MacLean A R; Journal of general Virology, (1993) November 74(Pt11) 2493-7.
9. Joany Chou, Bernard Roizman; Journal of Virology; (1990) March 1014-1020.
10. Rampling R, Cruickshank G, Papanastassiou V, et al. Gene Therapy 2000; 7(10):859-66.
11. Papanastassiou V, Rampling R, Fraser M, et al. Gene Therapy 2002; 9(6):398-406.
12. Harrow S, Papanastassiou V, Harland J, Mabbs R, Petty R, Fraser M, Hadley D, Brown SM and Rampling R. Gene Therapy In Press 2004.
13. Maclean A R, ul-Fareed M, Robertson L, Harland J, Brown S M, J Gen Virol 1991 March 72 (Pt 3):631-639.

1. An herpes simplex virus wherein the herpes simplex virus genome comprises nucleic acid encoding insulin.

2. An herpes simplex virus as claimed in claim 1 wherein said insulin is human insulin.

3. An herpes simplex virus as claimed in claim 1 or 2 wherein said nucleic acid comprises SEQ ID No.2 or SEQ ID No.5 or nucleic acid encoding the polypeptide of SEQ ID No.1 or SEQ ID No.6.

4. An herpes simplex virus as claimed in claim 1 wherein said nucleic acid has at least 60% sequence identity to SEQ ID No.2 or SEQ ID No.5 or to a nucleic acid encoding the polypeptide of SEQ ID No.1 or 6.

5. An herpes simplex virus as claimed in claim 4 wherein said degree of sequence identity is one of at least 70%, 80%, 85%, 90%, 95%, 96%, 97%, 98% or 99%.

6. An herpes simplex virus as claimed in claim 1 wherein said nucleic acid hybridises to the nucleic acid of SEQ ID No.2 or SEQ ID No.5, to the complement of SEQ ID No.2 or SEQ ID No.5 or to a nucleic acid encoding the polypeptide of SEQ ID No.1 or SEQ ID No.6 under high stringency conditions.

7. An herpes simplex virus as claimed in any one of claims 1 to 6 wherein said nucleic acid encodes a modified insulin polypeptide which incorporates one or more predetermined peptide motifs which are cleavage sites for a selected protease.

8. An herpes simplex virus as claimed in claim 7, wherein said selected protease does not cleave at least one of wild type preproinsulin, proinsulin or insulin.

9. An herpes simplex virus as claimed in claim 7 or 8 wherein a said predetermined peptide motif is a cleavage site for a protease present in cells which do not normally produce insulin and which may be infected by the herpes simplex virus.

10. An herpes simplex virus as claimed in any one of claims 7 to 9 wherein said peptide motif is formed by modification of a protease cleavage site of wild type insulin.

11. An herpes simplex virus as claimed in any one of claims 7 to 10 wherein a said peptide motif is formed by modification of the amino acid sequence of wild type insulin at which the C-peptide is cleaved.

12. An herpes simplex virus as claimed in any one of claims 7 to 11 wherein a said peptide motif is formed by modification of the amino acid sequence of wild type insulin at which the proinsulin polypeptide is cleaved into chains A and B.

13. An herpes simplex virus as claimed in any one of claims 7 to 12 wherein the selected protease is furin protease.

14. An herpes simplex virus as claimed in claim 13 wherein the peptide motif(s) recognised by furin protease is one or both of RTRR and RQKR.

15. An herpes simplex virus according to any one of claims 1 to 14 wherein said herpes simplex virus genome further comprises a regulatory nucleotide sequence operably linked to said nucleic acid encoding insulin, wherein said regulatory nucleotide sequence has a role in controlling transcription of said insulin.

16. An herpes simplex virus as claimed in claim 15 wherein said regulatory nucleotide sequence is inducible.

17. An herpes simplex virus as claimed in claim 15 or 16 wherein said regulatory nucleotide sequence is inducible in response to a change in the concentration of one or more selected metal ions.

18. An herpes simplex virus as claimed in any one of claims 15 to 17 wherein said regulatory nucleotide sequence is inducible in response to a change in the concentration of one or more selected carbohydrates.

19. An herpes simplex virus as claimed in any one of claims 15 to 18 wherein said regulatory nucleotide sequence comprises the human metallothionein promoter.

20. An herpes simplex virus as claimed in any one of claims 15 to 19 wherein said regulatory nucleotide sequence comprises a carbohydrate response element.

21. An herpes simplex virus as claimed in claim 20 wherein said carbohydrate response element has at least 80% sequence identity to SEQ ID No.7.

22. An herpes simplex virus as claimed in any preceding claim wherein the genome of the herpes simplex virus further comprises a marker nucleotide sequence.

23. An herpes simplex virus as claimed in any one of claims 1 to 22 wherein said nucleic acid is located in at least one RL1 locus of the herpes simplex virus genome.

24. An herpes simplex virus as claimed in any one of claims 1 to 23 wherein said nucleic acid is located in, or overlaps, at least one of the ICP34.5 protein coding sequences of the herpes simplex virus genome.

25. An herpes simplex virus as claimed in any one of claims 1 to 24 wherein the herpes simplex virus is a mutant of one of HSV-1 strains 17 or F or HSV-2 strain HG52.

26. An herpes simplex virus as claimed in any one of claims 1 to 24 wherein the herpes simplex virus is a mutant of HSV-1 strain 17 mutant 1716.

27. An herpes simplex virus as claimed in any one of claims 1 to 26 which is a gene specific null mutant.

28. An herpes simplex virus as claimed in any one of claims 1 to 27 which is an ICP34.5 null mutant.

29. An herpes simplex virus as claimed in any one of claims 1 to 26 which lacks at least one expressible ICP34.5 gene.

30. An herpes simplex virus as claimed in any one of claims 1 to 25 which lacks only one expressible ICP34.5 gene.

31. An herpes simplex virus as claimed in any one of claims 1 to 30 which is non-neurovirulent.

32. An herpes simplex virus as claimed in any one of claims 1 to 31 wherein said nucleic acid encoding insulin forms part of a nucleic acid cassette integrated in the genome of said herpes simplex virus, said cassette encoding:
(a) said nucleic acid encoding insulin; and nucleic acid encoding
(b) a ribosome binding site; and
(c) a marker,
wherein the nucleic acid encoding insulin is arranged upstream (5′) of the ribosome binding site and the ribosome binding site is arranged upstream (5′) of the marker.

33. An herpes simplex virus according to claim 32 wherein a regulatory nucleotide sequence is located upstream (5′) of the nucleic acid encoding insulin, wherein the regulatory nucleotide sequence has a role in regulating transcription of said nucleic acid encoding insulin.

34. An herpes simplex virus according to claim 32 or 33 wherein the cassette disrupts a protein coding sequence resulting in inactivation of the respective gene product.

35. An herpes simplex virus as claimed in any one of claims 32 to 34 wherein a transcription product of the cassette is a bi- or poly-cistronic transcript comprising a first cistron encoding insulin and a second cistron encoding the marker wherein the ribosome binding site is located between said first and second cistrons.

36. An herpes simplex virus as claimed in any one of claims 32 to 35 wherein the ribosome binding site comprises an internal ribosome entry site (IRES).

37. An herpes simplex virus as claimed in any one of claims 22 or 32 to 36 wherein the marker is a defined nucleotide sequence encoding a polypeptide.

38. An herpes simplex virus as claimed in claim 37 wherein the marker comprises the Green Fluorescent Protein (GFP) protein coding sequence or the enhanced Green Fluorescent Protein (EGFP) protein coding sequence.

39. An herpes simplex virus according to any one of claims 22 or 32 to 36 wherein the marker comprises a defined nucleotide sequence detectable by hybridisation under high stringency conditions with a corresponding labelled nucleic acid probe.

40. An herpes simplex virus as claimed in any one of claims 32 to 39 wherein the cassette further comprises nucleic acid encoding a polyadenylation sequence located downstream (3′) of the nucleic acid encoding the marker.

41. An herpes simplex virus as claimed in claim 39 wherein the polyadenylation sequence comprises the Simian Virus 40 (SV40) polyadenylation sequence.

42. An herpes simplex virus as claimed in any one of claims 1 to 41 for use in a method of medical treatment.

43. An herpes simplex virus as claimed in any one of claims 1 to 41 for use in the treatment of a disease state involving abnormal insulin expression or function.

44. An herpes simplex virus as claimed in any one of claims 1 to 41 for use in the treatment of diabetes.

45. Use of an herpes simplex virus as claimed in any one of claims 1 to 41 in the manufacture of a medicament for the treatment of diabetes.

46. A method of expressing one or more of preproinsulin, proinsulin and/or insulin in vitro or in vivo comprising the step of administering to a patient in need of treatment an herpes simplex virus as claimed in any one of claims 1 to 41.

47. A medicament, pharmaceutical composition or vaccine comprising an herpes simplex virus as claimed in any one of claims 1 to 41.

48. A medicament, pharmaceutical composition or vaccine as claimed in claim 47 further comprising a pharmaceutically acceptable carrier, adjuvant or diluent.

49. An herpes simplex virus, wherein the genome of said virus comprises a nucleic acid sequence encoding insulin in at least one of the long repeat regions (R_{L}).

50. An herpes simplex virus, wherein the genome of said virus comprises a nucleic acid sequence encoding insulin and wherein the herpes simplex virus is non-neurovirulent.

51. A composition comprising a herpes simplex virus according to claim 49 or claim 50.

52. An herpes simplex virus for use in the treatment of a disease state involving abnormal insulin expression or function, wherein the genome of said virus comprises a nucleic acid sequence encoding insulin in at least one of the long repeat regions (R_{L}).

53. An herpes simplex virus for use in the treatment of a disease state involving abnormal insulin expression or function, wherein the genome of said virus comprises a nucleic acid sequence encoding insulin and wherein the herpes simplex virus is non-neurovirulent.

54. Use of an herpes simplex virus, wherein the genome of said virus comprises a nucleic acid sequence encoding insulin in at least one of the long repeat regions (R_{L}), in the manufacture of a medicament for the treatment of a disease state involving abnormal insulin expression or function.

55. Use of an herpes simplex virus, wherein the genome of said virus comprises a nucleic acid sequence encoding insulin and wherein the herpes simplex virus is non-neurovirulent, in the manufacture of a medicament for the treatment of a disease state involving abnormal insulin expression or function.

56. A method of expressing insulin in vitro or in vivo, said method comprising the step of infecting at least one cell or tissue of interest with a herpes simplex virus, wherein the genome of said virus comprises a nucleic acid sequence encoding insulin in at least one of the long repeat regions (R_{L}), said insulin operably linked to a transcription regulatory sequence.

57. A method of expressing insulin in vitro or in vivo, said method comprising the step of infecting at least one cell or tissue of interest with a non-neurovirulent herpes simplex virus, wherein the genome of said virus comprises a nucleic acid sequence encoding insulin, said insulin operably linked to a transcription regulatory sequence.

58. A cell, in vitro, infected with a herpes simplex virus according to any one of claims 1 to 41.


SEQUENCE LISTING (9 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - DNA, 450 bp, Homo sapiens
gctgcatcagaagaggccatcaagcacatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccccacccgccgcctcctgcaccgagagagatggaataaagcccttgaaccagc

SEQ ID NO: 3 - DNA, 28 bp, Artificial sequence
  Reverse mutagenic primer
gagcttccaccaggtctgagccgcacag

SEQ ID NO: 4 - DNA, 28 bp, Artificial sequence
  Forward mutagenic primer
ctgtgcggctcagacctggtggaagctc

SEQ ID NO: 5 - DNA, 333 bp, Artificial sequence
  Mutant human proinsulin (HisB10Asp proinsulin)
  CDS (1)..(333)
atggccctgtggatgcgcctcctgcccctgctggcgctgctggccctctggggacctgacccagccgcagcctttgtgaaccaacacctgtgcggctcagacctggtggaagctctctacctagtgtgcggggaacgaggcttcttctacacacccaggacccgccgggaggcagaggacctgcaggtggggcaggtggagctgggcgggggccctggtgcaggcagcctgcagcccttggccctggaggggtcccggcagaagcgtggcattgtggaacaatgctgtaccagcatctgctccctctaccagctggagaactactgcaactag

SEQ ID NO: 6 - protein, 110 aa, Artificial sequence
  Mutant human proinsulin (HisB10Asp proinsulin)
MALWMRLLPLLALLALWGPDPAAAFVNQHLCGSDLVEALYLVCGERGFFYTPRTRREAEDLQVGQVELGGGPGAGSLQPLALEGSRQKRGIVEQCCTSICSLYQLENYCN

SEQ ID NO: 7 - DNA, 95 bp, Artificial sequence
  Nucleotide sequence of carbohydrate response element
agttctgccacgtgcttgagttctcacgtggtggccacgtgcttgagttctcacgtggggccacgtgcttgagttctcacgtggtggccacgtac

SEQ ID NO: 8 - protein, 4 aa, Artificial sequence
  Cleavage site for furin protease
RTRR

SEQ ID NO: 9 - protein, 4 aa, Artificial sequence
  Cleavage site for furin protease
RQKR